SEQUENCE LISTING (2 sequences)

SEQ ID NO: 1 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Artificial Sequence
  Description of Artificial Sequence Synthetic construct
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

(19) United States
(12) Patent Application Publication
(10) Pub. No.: US 2007/0092911 A1
(43) Pub. Date: Apr. 26, 2007

(54) Methods and compositions for diagnosis and /or prognosis in systemic inflammatory response syndromes

(76) Inventors: Kenneth F. Buechler, Rancho Santa Fe, CA (US); Joseph M. Anderberg, Encinitas, CA (US); Paul H. McPherson, Encinitas, CA (US)

Correspondence Address: Barry S. Wilson;FOLEY & LARDNER LLP, P.O. Box 80278, San Diego, CA, 92138-0278, US

(21) Appl. No.: 11/543,312

(22) Filed: Oct. 3, 2006

Related U.S. Application Data
(60) Provisional application No. 60/723,194, filed on Oct. 3, 2005.
(60) Provisional application No. 60/801,485, filed on May 17, 2006.
(60) Provisional application No. 60/763,830, filed on Jan. 31, 2006.
(60) Provisional application No. 60/736,992, filed on Nov. 14, 2005.
(60) Provisional application No. 60/831,604, filed on Jul. 17, 2006.

(51) Int. Cl. G01N 33/53

(52) U.S. Cl. 435/7.1

(57) ABSTRACT

The present invention relates to methods and compositions for symptom-based differential diagnosis, prognosis, and determination of treatment regimens in subjects. In particular, the invention relates to methods and compositions selected to rule in or out SIRS, or for differentiating sepsis, severe sepsis, septic shock and/or MODS from each other and/or from non-infectious SIRS.

### CROSS-REFERENCE TO RELATED PATENT APPLICATIONS

This application claims the benefit under 35 U.S.C § 119(e) of U.S. patent Application Ser. No. 60/723,194, filed Oct. 3, 2005, Ser. No. 60/736,992, filed Nov. 14, 2005, Ser. No. 60/763,830, filed Jan. 31, 2006, Ser. No. 60/801,485, filed May 17, 2006, and Ser. No. 60/831,604, filed Jul. 17, 2006, each of which is incorporated by reference herein in its entirety including all figures and tables.

### FIELD OF THE INVENTION

The present invention relates to the identification and use of diagnostic markers related to sepsis. In a various aspects, the invention relates to methods and compositions for use in assigning a treatment pathway to subjects suffering from SIRS, sepsis, severe sepsis, septic shock and/or multiple organ dysfunction syndrome.

### BACKGROUND OF THE INVENTION

The following discussion of the background of the invention is merely provided to aid the reader in understanding the invention and is not admitted to describe or constitute prior art to the present invention.

The term “sepsis” has been used to describe a variety of clinical conditions related to systemic manifestations of inflammation accompanied by an infection. Because of clinical similarities to inflammatory responses secondary to non-infectious etiologies, identifying sepsis has been a particularly challenging diagnostic problem. Recently, the American College of Chest Physicians and the American Society of Critical Care Medicine (Bone et al., Chest 101: 1644-53, 1992) published definitions for “Systemic Inflammatory Response Syndrome” (or “SIRS”), which refers generally to a severe systemic response to an infectious or non-infectious insult, and for the related syndromes “sepsis,” “severe sepsis,” and “septic shock,” and extending to multiple organ dysfunction syndrome (“MODS”). These definitions, described below, are intended for each of these phrases for the purposes of the present application.

“SIRS” refers to a condition that exhibits two or more of the following:

a temperature >38° C. or <36° C.;

a heart rate of >90 beats per minute (tachycardia);

a respiratory rate of >20 breaths per minute (tachypnea) or a PₐCO₂<4.3 kPa; and

a white blood cell count >12,000 per mm³, <4,000 per mm³, or >10% immature (band) forms.

“Sepsis” refers to SIRS, further accompanied by a clinically evident or microbiologically confirmed infection. This infection may be bacterial, fungal, parasitic, or viral.

“Severe sepsis” refers to sepsis, further accompanied by organ hypoperfusion made evident by at least one sign of organ dysfunction such as hypoxemia, oliguria, metabolic acidosis, or altered cerebral function.

“Septic shock” refers to severe sepsis, further accompanied by hypotension, made evident by a systolic blood pressure <90 mm Hg, or the requirement for pharmaceutical intervention to maintain blood pressure.

MODS (multiple organ dysfunction syndrome) is the presence of altered organ function in a patient who is acutely ill such that homeostasis cannot be maintained without intervention. Primary MODS is the direct result of a well-defined insult in which organ dysfunction occurs early and can be directly attributable to the insult itself. Secondary MODS develops as a consequence of a host response and is identified within the context of SIRS.

A systemic inflammatory response leading to a diagnosis of SIRS may be related to both infection and to numerous non-infective etiologies, including burns, pancreatitis, trauma, heat stroke, and neoplasia. While conceptually it may be relatively simple to distinguish between sepsis and non-septic SIRS, no diagnostic tools have been described to unambiguously distinguish these related conditions. See, e.g., Llewelyn and Cohen, Int. Care Med. 27: S10-S32, 2001. For example, because more than 90% of sepsis cases involve bacterial infection, the “gold standard” for confirming infection has been microbial growth from blood, urine, pleural fluid, cerebrospinal fluid, peritoneal fluid, synnovial fluid, sputum, or other tissue specimens. Such culture has been reported, however, to fail to confirm 50% or more of patients exhibiting strong clinical evidence of sepsis. See, e.g., Jaimes et al., Int. Care Med 29: 1368-71, published electronically Jun. 26, 2003.

The physiologic responses leading to the systemic manifestations of inflammation in sepsis remain unclear. Activation of immune cells occurs in response to the LPS endotoxin of gram negative bacteria and exotoxins of gram positive bacteria. This activation leads to a cascade of events mediated by proinflammatory cytokines, adhesion molecules, vasoactive mediators, and reactive oxygen species. Various organs, including the liver, lungs, heart, and kidney are affected directly or indirectly by this cascade. Sepsis is also associated with disseminated intravascular coagulation (“DIC”), mediated presumably by cytokine activation of coagulation. Fluid and electrolyte balance are also affected by increases in capillary perfusion and reduced oxygenation of tissues. Unchecked, the uncontrolled inflammatory response created can lead to ischemia, loss of organ function, and death.

Despite the availability of antibiotics and supportive therapy, sepsis represents a significant cause of morbidity and mortality. A recent study estimated that 751,000 cases of severe sepsis occur in the United States annually, with a mortality rate of from 30-50%. Angus et al., Crit. Care Med. 29: 1303-10, 2001. Recently, an organization of medical care groups referred to as the “Surviving Sepsis Campaign” issued guidelines for managing subjects suffering from severe sepsis and septic shock. Dellinger et al., Crit. Care Med. 32: 858-873, 2004. These guidelines draw from, amongst other sources, the “Early Goal Directed Therapy” therapy regimen developed by Rivers and colleagues. See, e.g., New Engl. J. Med. 345: 1368-77. 2001.

Several laboratory tests have been investigated or proposed for use, in conjunction with a complete clinical examination of a subject, for the diagnosis and prognosis of sepsis. See, e.g., U.S. Pat. Nos. 5,639,617 and 6,303,321; Patent publications US2005/0196817, WO2005/048823, WO2004/046181, WO2004/043236, US2005/0164238; and Charpentier et al., Crit. Care Med. 32: 660-65, 2004; Castillo et al., Int. J. Infect. Dis. 8: 271-74, 2004; Chua and Kang-Hoe, Crit. Care 8: R248-R250, 2004; Witthaut et al., Int. Care Med. 29: 1696-1702, 2003; Jones and Kline, Ann. Int. Med. 42: 714-15, 2003; Maeder et al., Swiss Med. Wkly. 133: 515-18, 2003; Giamarellos-Bourboulis et al., Intensive Care Med. 28: 1351-56, 2002; Harbarth et al., Am. J. Respir. Crit. Care Med. 164: 396-402, 2001; Martin et al., Pediatrics 108: (4) e61 1-6, 2001; and Bossink et al., Chest 113: 1533-41, 1998.

### BRIEF SUMMARY OF THE INVENTION

The present invention relates to the identification and use of markers for the detection of sepsis, the differentiation of sepsis from other causes of SIRS, and in the stratification of risk in sepsis patients. The methods and compositions of the present invention can be used to facilitate the treatment of patients and the development of additional diagnostic and/or prognostic indicators and therapies.

In various aspects, the invention relates to materials and procedures for identifying markers that may be used to direct therapy in subjects; to using such markers in treating a patient and/or to monitor the course of a treatment regimen; to using such markers to identify subjects at risk for one or more adverse outcomes related to SIRS; and for screening compounds and pharmaceutical compositions that might provide a benefit in treating or preventing such conditions.

In a first aspect, the invention relates to diagnostic methods for identifying a subject suffering from SIRS, sepsis, severe sepsis, septic shock and/or MODS, and/or for distinguishing amongst these conditions. These methods comprise analyzing a test sample or test samples obtained from a subject for the presence or amount of one or more markers selected from the group consisting of adiponectin, adrenomedullin, angiotensinogen, apolipoprotein C1, big endothelin-1, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL5, CXCL9, CXCL13, CXCL16, CXCL6, cystatin C, D-Dimer, sDR6, glutathione-S-transferase A, HMG-1, intestinal fatty acid binding protein, liver fatty acid-binding protein, IGFBP-1, IL-10, IL-1β, interleukin-1 receptor antagonist (IL-1RA), IL-22, IL-2sRa, IL-6, IL-8, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TNF-α, TNF-R1a, TNF-sR14, sTNFRSF3, sTNFRSF7, sTNFRSF11A, TREM-1, TREM-1sv, UCRP, uPAR, and VCAM-1, or markers related thereto. The term “related markers” is defined hereinafter. Preferred panels comprise measuring at least one, preferably at least two, more preferably at least three, still more preferably at least four, yet more preferably at least five, and most preferably at least six or more of the above markers. Other markers that may be used together with one or more of these markers are described hereinafter, particularly in the examples. These other markers are preferably selected from the group consisting of markers related to blood pressure regulation, markers related to coagulation and hemostasis, markers related to apoptosis, and/or markers related to inflammation. The results of the analysis, in the form of assay results, are correlated to the presence or absence of SIRS, sepsis, severe sepsis, septic shock and/or MODS, and/or may differentiate between one or more of these conditions.

In a related aspect, the invention relates to methods for determining a prognosis for a subject. These methods similarly comprise analyzing a test sample or test samples obtained from a subject for the presence or amount of one or more markers selected from the group consisting of adiponectin, adrenomedullin, angiotensinogen, apolipoprotein C1, big endothelin-1, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL5, CXCL9, CXCL13, CXCL16, CXCL6, cystatin C, D-Dimer, sDR6, glutathione-S-transferase A, HMG-1, intestinal fatty acid binding protein, liver fatty acid-binding protein, IGFBP-1, IL-10, IL-1β, interleukin-1 receptor antagonist (IL-1RA), IL-22, IL-2sRa, IL-6, IL-8, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TNF-α, TNF-R1a, TNF-sR14, sTNFRSF3, sTNFRSF7, sTNFRSF11A, TREM-1, TREM-1sv, UCRP, uPAR, and VCAM-1, or markers related thereto. Preferred panels comprise measuring at least one, preferably at least two, more preferably at least three, still more preferably at least four, yet more preferably at least five, and most preferably at least six or more of the above markers. Other markers that may be used together with one or more of these markers are described hereinafter, particularly in the examples. These other markers are preferably selected from the group consisting of markers related to blood pressure regulation, markers related to coagulation and hemostasis, markers related to apoptosis, and/or markers related to inflammation. The results of the analysis, in the form of assay results, are correlated to the likelihood of a future outcome, either positive (e.g., that the subject is likely to live) or negative (e.g., that the subject is at an increased risk of death).

Preferred methods for these two related aspects comprise performing one or more assays that are configured to detect one or more of adiponectin, adrenomedullin, angiotensinogen, apolipoprotein C1, big endothelin-1, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL5, CXCL9, CXCL13, CXCL16, CXCL6, cystatin C, D-Dimer, sDR6, glutathione-S-transferase A, HMG-1, intestinal fatty acid binding protein, liver fatty acid-binding protein, IGFBP-1, IL-10, IL-1β, interleukin-1 receptor antagonist (IL-1RA), IL-22, IL-2sRa, IL-6, IL-8, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TNF-α, TNF-R1a, TNF-sR14, sTNFRSF3, sTNFRSF7, sTNFRSF11A, TREM-1, TREM-1sv, UCRP, uPAR, VCAM-1. Preferred panels comprise measuring at least one, preferably at least two, more preferably at least three, still more preferably at least four, yet more preferably at least five, and most preferably at least six or more of the above markers. As noted above, assays configured to detect one or more other markers that may be used together with one or more of these assays are described hereinafter. These other markers are preferably selected from the group consisting of markers related to blood pressure regulation, markers related to coagulation and hemostasis, markers related to apoptosis, and/or markers related to inflammation.

In certain embodiments, a plurality of markers, comprising 2, 3, 4, 5, 6, 7, 8, 9, 10, 15, 20, or more or individual markers, are combined into a marker panel. While such panels may be composed of entirely of markers selected from the group consisting of adiponectin, adrenomedullin, angiotensinogen, apolipoprotein C1, big endothelin-1, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL5, CXCL9, CXCL13, CXCL16, CXCL6, cystatin C, D-Dimer, sDR6, glutathione-S-transferase A, HMG-1, intestinal fatty acid binding protein, liver fatty acid-binding protein, IGFBP-1, IL-10, IL-1β, interleukin-1 receptor antagonist (IL-1RA), IL-22, IL-2sRa, IL-6, IL-8, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TNF-α, TNF-R1a, TNF-sR14, sTNFRSF3, sTNFRSF7, sTNFRSF11A, TREM-1, TREM-1sv, UCRP, uPAR, and VCAM-1, or markers related thereto, additional markers may be included in such panels. Exemplary additional markers are described in detail hereinafter.

Preferred panels comprise measuring at least one, preferably at least two, more preferably at least three, still more preferably at least four, yet more preferably at least five, and most preferably at least six or more of the following markers: BNP, NT-proBNP, CCL19, CXCL5, CXCL9, cystatin C, D-dimer, L-FABP, myeloperoxidase, myoglobin, NGAL, sTNFRSF3, sTNFRSF7, sTNFRSF11A, active protein C, latent protein C, total protein C, and UCRP, or markers related thereto. And preferred methods comprise performing assays that are configured to detect at least one, preferably at least two, more preferably at least three, still more preferably at least four, yet more preferably at least five, and most preferably at least six or more of the following markers: BNP, NT-proBNP, CCL19, CXCL5, CXCL9, cystatin C, D-dimer, L-FABP, myeloperoxidase, myoglobin, NGAL, sTNFRSF3, sTNFRSF7, sTNFRSF11A, active protein C, latent protein C, total protein C, and UCRP. Other markers not in this list may be included in such panels. Exemplary additional markers to optionally include in such preferred panels are described in detail herein.

Another preferred method comprises performing one or more immunoassays to detect a plurality of markers, provided that at least two of said plurality of markers detected is selected from the group consisting of NT-proBNP, proBNP, BNP₇₉₋₁₀₈, BNP, BNP₃₋₁₀₈, CCL19, CCL23, CRP, cystatin C, D-dimer, IL-1ra, IL-2sRa, myeloperoxidase, myoglobin, NGAL, lymphotoxin B receptor, peptidoglycan recognition protein, procalcitonin, procarboxypeptidase B, active protein C, latent protein C, total protein C, and sTNFR1a. In certain embodiments, the assay method further comprises performing one or more additional immunoassays that detect one or more additional markers other than those listed above in this paragraph. One or more variables that are not immunoassay results may be used together with one or more of these markers. The variables that are not immunoassay results comprise one or more of heart rate, temperature, respiration rate, white blood cell count, blood gas level, venous blood pH, blood lactate level, renal function, electrolyte level, blood pressure, pulmonary wedge pressure, or blood culture result.

Yet another preferred method comprises performing at least two, more preferably at least three, still more preferably at least four, yet more preferably at least five immunoassays that detect markers selected from the group consisting of NT-proBNP, proBNP, BNP₇₉₋₁₀₈, BNP, BNP₃₋₁₀₈, CCL23, CRP, D-dimer, IL-1ra, NGAL, peptidoglycan recognition protein, active protein C, latent protein C, total protein C, and sTNFR1a.

Still another preferred method comprises performing an immunoassay that detects one or more of BNP, proBNP, NT-proBNP, or BNP₃₋₁₀₈, an immunoassay that detects one or more of active protein C, latent protein C, total protein C, and at least one immunoassay that detects a marker selected from the group consisting of CCL23, CRP, D-dimer, IL-1ra, NGAL, peptidoglycan recognition protein, and sTNFR1a.

Another preferred method comprises performing an immunoassay that detects one or more of BNP, proBNP, NT-proBNP, or BNP₃₋₁₀₈, at least one immunoassay that detects a marker selected from the group consisting of C-reactive protein, D-dimer, and IL-1ra, and at least one immunoassay that detects a marker selected from the group consisting of CCL23, peptidoglycan recognition protein, and sTNFR1a.

Yet another preferred method comprises performing an immunoassay that detects peptidoglycan recognition protein and an immunoassay that detects sTNFR1a.

In another aspect, the invention relates to diagnostic methods for identifying a subject suffering from SIRS, sepsis, severe sepsis, septic shock and/or MODS. These methods comprise analyzing a test sample or test samples obtained from a subject for the presence or amount of one or more markers selected from the group consisting of LIGHT, CCL16, and MMP7, or markers related thereto. The term “related markers” is defined hereinafter. The results of the analysis, in the form of assay results, are correlated to the presence or absence of SIRS, sepsis, severe sepsis, septic shock and/or MODS, and/or may differentiate between one or more of these conditions. Preferred assays are configured to detect LIGHT, CCL16, and/or MMP7.

In a related aspect, the invention relates to methods for determining a prognosis for a subject suffering from SIRS, sepsis, severe sepsis, septic shock and/or MODS. These methods similarly comprise analyzing a test sample or test samples obtained from a subject for the presence or amount of one or more markers selected from the group consisting of LIGHT, CCL16, and MMP7, or markers related thereto. The results of the analysis, in the form of assay results, are correlated to the likelihood of a future outcome, either positive (e.g., that the subject is likely to live) or negative (e.g., that the subject is at an increased risk of death).

In a further aspect, there is provided a method of diagnosing SIRS, sepsis, severe sepsis, septic shock, or MODS in a subject, or assigning a prognostic risk for one or more clinical outcomes for a subject suffering from SIRS, sepsis, severe sepsis, septic shock, or MODS, the method comprising:

performing an assay method on one or more samples obtained from said subject, wherein said assay method comprises performing one or more immunoassays to detect a plurality of markers, provided that at least two of said plurality of markers detected is selected from the group consisting of NT-proBNP, proBNP, BNP₇₉₋₁₀₈, BNP, BNP₃₋₁₀₈, CCL19, CCL23, CRP, cystatin C, D-dimer, IL-1ra, IL-2sRa, myeloperoxidase, myoglobin, NGAL, lymphotoxin B receptor, peptidoglycan recognition protein, procalcitonin, procarboxypeptidase B, active protein C, latent protein C, total protein C, and sTNFR1a; and

relating the immunoassay results obtained from said assay method to one or more diagnoses or prognoses selected from the group consisting of the presence or absence of SIRS, the presence or absence of sepsis, the presence or absence of severe sepsis, the presence or absence of septic shock, and the prognostic risk of one or more clinical outcomes for the subject suffering from or believed to suffer from SIRS, sepsis, severe sepsis, septic shock, or MODS.

As described above, a plurality of markers, comprising 2, 3, 4, 5, 6, 7, 8, 9, 10, 15, 20, or more or individual markers, are combined into a marker panel. While panels may be composed of entirely of markers selected from the group consisting of LIGHT, CCL16, and MMP7, or markers related thereto, additional markers may be included in such panels. Exemplary additional markers are described in detail hereinafter. Preferred markers for inclusion in such marker panels include those markers related to blood pressure regulation, markers related to coagulation and hemostasis, markers related to apoptosis, and/or markers related to inflammation.

In certain embodiments, concentrations of the individual markers can each be compared to a level (a “threshold”) that is preselected to rule in or out one or more particular diagnoses, prognoses, and/or therapy regimens. In these embodiments, correlating of each of the subject's selected marker level can comprise comparison to thresholds for each marker of interest that are indicative of a particular diagnosis. Similarly, by correlating the subject's marker levels to prognostic thresholds for each marker, the probability that the subject will suffer one or more future adverse outcomes may be determined.

In other embodiments, particular thresholds for one or more markers in a panel are not relied upon to determine if a profile of marker levels obtained from a subject are correlated to a particular diagnosis or prognosis. Rather, the present invention may utilize an evaluation of the entire profile of markers to provide a single result value (e.g., a “panel response” value expressed either as a numeric score or as a percentage risk). In such embodiments, an increase, decrease, or other change (e.g., slope over time) in a certain subset of markers may be sufficient to indicate a particular condition or future outcome in one patient, while an increase, decrease, or other change in a different subset of markers may be sufficient to indicate the same or a different condition or outcome in another patient. Methods for performing such analyses are described hereinafter.

In yet other embodiments, multiple determinations of one or more markers can be made, and a temporal change in the markers can be used to rule in or out one or more particular diagnoses and/or prognoses. For example, one or more markers may be determined at an initial time, and again at a second time, and the change (or lack thereof) in the marker level(s) over time determined. In such embodiments, an increase in the marker from the initial time to the second time may be indicative of a particular prognosis, of a particular diagnosis, etc. Likewise, a decrease in the marker from the initial time to the second time may be indicative of a particular prognosis, of a particular diagnosis, etc. In such a panel, the markers need not change in concert with one another. Temporal changes in one or more markers may also be used together with single time point marker levels to increase the discriminating power of marker panels. In yet another alternative, a “panel response” may be treated as a marker, and temporal changes in the panel response may be indicative of a particular prognosis, diagnosis, etc.

As discussed in detail herein, preferably a plurality of markers may be combined to increase the predictive value of the analysis in comparison to that obtained from the markers individually. Such panels may comprise 2, 3, 4, 5, 6, 7, 8, 9, 10, 15, 20, or more or individual markers. The skilled artisan will also understand that diagnostic markers, differential diagnostic markers, prognostic markers, time of onset markers, etc., may be combined in a single assay or device. For example, certain markers measured by a device or instrument may be used provide a prognosis, while a different set of markers measured by the device or instrument may rule in and/or out particular therapies; each of these sets of markers may comprise unique markers, or may include markers that overlap with one or both of the other sets. Markers may also be commonly used for multiple purposes by, for example, applying a different set of analysis parameters (e.g., different midpoint, linear range window and/or weighting factor) to the marker(s) for the different purpose(s).

In certain embodiments, one or more markers are correlated to a therapy, prognosis, condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

While exemplary panels are described herein, one or more markers may be replaced, added, or subtracted from these exemplary panels while still providing clinically useful results. Panels may comprise both specific markers of a disease (e.g., markers that are increased or decreased in bacterial infection, but not in other disease states) and/or non-specific markers (e.g., markers that are increased or decreased due to inflammation, regardless of the cause; markers that are increased or decreased due to changes in hemostasis, regardless of the cause, etc.). While certain markers may not individually be definitive in the methods described herein, a particular “fingerprint” pattern of changes may, in effect, act as a specific indicator of disease state. As discussed above, that pattern of changes may be obtained from a single sample, or may optionally consider temporal changes in one or more members of the panel (or temporal changes in a panel response value).

In addition to one or more markers selected from the group consisting of sTNFRSF3, sTNFRSF7, sTNFRSF11A, LIGHT, CCL16, CXCL5, CXCL9, MMP7, adiponectin, adrenomedullin, angiotensinogen, apolipoprotein C1, big endothelin-1, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL13, CXCL16, CXCL6, cystatin C, D-Dimer, sDR6, glutathione-S-transferase A, HMG-1, intestinal fatty acid binding protein, IGFBP-1, IL-10, IL-1β, IL-1RA, IL-22, IL-2sRa, IL-6, IL-8, L-FABP, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TNF-α, TNF-R1a, TNF-sR14, TREM-1, TREM-1sv, uPAR, UCRP, and VCAM-1, or markers related thereto, preferred marker panels can comprise, for example, one or more other marker(s) selected from the following groups:

one or more markers selected from the group consisting of atrial natriuretic peptide (“ANP), NT-proANP, pro-ANP, NT-pro BNP, pro-BNP, C-type natriuretic peptide, NT-proCNP, pro-CNP, urotensin II, arginine vasopressin, aldosterone, angiotensin I, angiotensin II, angiotensin III, bradykinin, procalcitonin, calcitonin gene related peptide, calcyphosine, endothelin-2, endothelin-3, renin, and urodilatin, or markers related thereto (referred to collectively as “markers related to blood pressure regulation”);

and/or one or more markers selected from the group consisting of acute phase reactants, cell adhesion molecules such as soluble intercellular adhesion molecule-1 (“sICAM-1”), soluble intercellular adhesion molecule-2 (“sICAM-2”), soluble intercellular adhesion molecule-3 (“sICAM-3”), other interleukins, other chemokines in the CXCL and CCL families, lipocalin-type prostaglandin D synthase, mast cell tryptase, eosinophil cationic protein, KL-6, haptoglobin, tumor necrosis factor β, soluble Fas ligand, soluble Fas (Apo-1), TRAIL, TWEAK, fibronectin, and vascular endothelial growth factor (“VEGF”), or markers related thereto (referred to collectively as “markers related to inflammation”);

and/or one or more markers selected from the group consisting of plasmin, fibrinogen, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, and thrombus precursor protein, or markers related thereto (referred to collectively as “markers related to coagulation and hemostasis”);

and/or one or more marker(s) selected from the group consisting of spectrin, cathepsin D, cytochrome c, s-acetyl glutathione, and ubiquitin fusion degradation protein 1 homolog, or markers related thereto (referred to collectively as “markers related to apoptosis”).

Other markers within each of these general classes will be known to those of skill in the art.

In addition to those “markers related to inflammation,” one or more markers related to inflammation may also be selected from the group of acute phase reactants consisting of hepcidin, HSP-60, HSP-65, HSP-70, asymmetric dimethylarginine (an endogenous inhibitor of nitric oxide synthase), matrix metalloproteins 11 and 3, defensin HBD 1, defensin HBD 2, serum amyloid A, oxidized LDL, insulin like growth factor, transforming growth factor β, inter-α-inhibitors, e-selectin, hypoxia-inducible factor-1α, inducible nitric oxide synthase (“I-NOS”), intracellular adhesion molecule, lactate dehydrogenase, n-acetyl aspartate, prostaglandin E2, receptor activator of nuclear factor and (“RANK”) ligand, or markers related thereto. Other markers within the general class of acute phase reactants will be known to those of skill in the art.

Additionally, one or more markers related to reactive oxygen species may also be measured as part of such a panel. The marker(s) may be selected from the group consisting of superoxide dismutase, glutathione, α-tocopherol, ascorbate, inducible nitric oxide synthase, lipid peroxidation products, nitric oxide, and breath hydrocarbons (preferably ethane), or markers related thereto.

Additional markers and/or marker classes may be utilized for such panels to provide further ability to discriminate amongst diseases. For example, the inflammatory response and resulting effects on capillaries and reduced oxygenation of tissues implicate one or more markers related to the acute phase response, one or more markers related to vascular tissues, and one or more tissue-specific markers (e.g., neural-specific markers such as S100β), the levels of which are increased in ischemic conditions. Thus, one or more markers selected from the group consisting of α-2 actin, basic calponin 1, β-1 integrin, acidic calponin, caldesmon, cysteine rich protein-2 (“CRP 2” or “CSRP 2”), elastin, fibrillin 1, latent transforming growth factor beta binding protein 4 (“LTBP 4”), smooth muscle myosin, smooth muscle myosin heavy chain, and transgelin, or markers related thereto (referred to collectively as “markers related to vascular tissue”) may be included in such a panel. Additional markers and marker classes are described hereinafter.

Preferred panels for the diagnosis of one or more conditions within the diagnosis of SIRS, and/or prognosis of one or more conditions within the diagnosis of SIRS, and/or for differentiating conditions within the diagnosis of SIRS, comprise performing assays configured to detect at least one, preferably at least two, more preferably at least three, still more preferably at least four, yet more preferably at least five, and most preferably at least six or more of the following markers: adrenomedullin, big endothelin-1, BNP, proBNP, NT-proBNP, CCL5, CCL19, CCL23, CK-MB, complement C3a, creatinine, CXCL13, CXCL16, cystatin C, D-dimer, HSP-60, sICAM-1, IL-1ra, IL-2sRA, IL-6, IL-10, lactate, MCP-1, myoglobin, myeloperoxidase, NGAL, procalcitonin, active protein C, latent protein C, total protein C, serum amyloid A, tissue factor, TNF-R1a, TREM-1, sTNFRSF11A, TIMP-1, and uPAR, or markers related thereto; and at least one, preferably at least two, more preferably at least three, still more preferably at least four, yet more preferably at least five, and most preferably at least six or more of the following markers: adiponectin, angiotensinogen, apolipoprotein C1, CCL20, CXCL5, CXCL9, L-FABP, placental growth factor, sTNFRSF3, sTNFRSF7, and UCRP, or markers related thereto.

In a related aspect, the present invention relates to methods for identifying marker panels for use in the foregoing methods. In developing a panel of markers useful in diagnosis, prognosis, and/or therapy, data for a number of potential markers may be obtained from a group of subjects by testing for the presence or level of certain markers. The group of subjects may then be divided into sets. For example, a first set includes subjects who have been confirmed as having a disease or, more generally, being in a first condition state. The confirmation of this condition state may be made through a more rigorous and/or expensive testing, such as culture of a tissue sample for organisms in sepsis. Hereinafter, subjects in this first set will be referred to as “diseased”. A second set of subjects is selected from those who do not fall within the first set. Subjects in this second set will hereinafter be referred to as “non-diseased”.

The data obtained from subjects in these sets includes levels of a plurality of markers. Preferably, data for the same set of markers is available for each patient. Exemplary markers are described herein. Actual known relevance of the marker(s) to the disease of interest is not required. Methods for comparing these subject sets for relevance of one or more markers is described hereinafter. Embodiments of the methods and systems described herein may be used to determine which of the candidate markers are most relevant to the diagnosis of the disease or condition or of a given prognosis.

In yet a further aspect, the invention relates to devices to perform one or more of the methods described herein. Such devices preferably contain a plurality of diagnostic zones, each of which is related to a particular marker of interest. Such diagnostic zones are preferably discrete locations within a single assay device. Such devices may be referred to as “arrays” or “microarrays.” Following reaction of a sample with the devices, a signal is generated from the diagnostic zone(s), which may then be correlated to the presence or amount of the markers of interest. Numerous suitable devices are known to those of skill in the art.

### DETAILED DESCRIPTION OF THE INVENTION

The present invention relates to methods and compositions for symptom-based differential diagnosis, prognosis, and determination of treatment regimens in subjects. In particular, the invention relates to methods and compositions selected to rule in or out SIRS, or for differentiating sepsis, severe sepsis, septic shock, and/or MODS from each other and/or from non-infectious SIRS.

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. In the case of SIRS, the condition exists, by definition, whenever two or more of the following symptoms are present:

a temperature >38° C. or <36° C.;

a heart rate of >90 beats per minute (tachycardia);

a respiratory rate of >20 breaths per minute (tachypnea) or a PₐCO₂<4.3 kPa; and

a white blood cell count >12,000 per mm³, <4,000 per mm³, or >10% immature (band) forms.

The present invention describes methods and compositions that can assist in the differential diagnosis of one or more nonspecific symptoms by providing diagnostic markers that are designed to rule in or out one, and preferably a plurality, of possible etiologies for the observed symptoms. Symptom-based differential diagnosis described herein can be achieved using panels of diagnostic markers designed to distinguish between possible diseases that underlie a nonspecific symptom observed in a patient.

Definitions

The term “therapy regimen” refers to one or more interventions made by a caregiver in hopes of treating a disease or condition. The term “early sepsis therapy regimen” refers to a set of supportive therapies designed to reduce the risk of mortality when administered within the initial 24 hours, more preferably within the initial 12 hours, and most preferably within the initial 6 hours or earlier, of assigning a diagnosis of SIRS, sepsis, severe sepsis, septic shock, or MODS to a subject. Such supportive therapies comprise a spectrum of treatments including resuscitation, fluid delivery, vasopressor administration, inotrope administration, steroid administration, blood product administration, and/or sedation. See, e.g., Dellinger et al., Crit. Care Med. 32: 858-873, 2004, and Rivers et al., N. Engl. J. Med. 345: 1368-1377, 2001 (providing a description of “early goal directed therapy” as that term is used herein), each of which is hereby incorporated by reference. Preferably, such an early sepsis therapy regimen comprises one or more, and preferably a plurality, of the following therapies:

maintenance of a central venous pressure of 8-12 mm Hg, preferably by administration of crystalloids and/or colloids as necessary;

maintenance of a mean arterial pressure of ≧65 mm Hg, preferably by administration of vasopressors and/or vasodilators as necessary;

maintenance of a central venous oxygen saturation of ≧70%, preferably by administration of transfused red blood cells to a hematocrit of at least 30% and/or administration of dobutamine as necessary; and

administration of mechanical ventilation as necessary.

The term “marker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments. Markers can also include clinical “scores” such as a pre-test probability assignment, a pulmonary hypertension “Daniel” score, an NIH stroke score, a Sepsis Score of Elebute and Stoner, a Duke Criteria for Infective Endocarditis, a Mannheim Peritonitis Index, an “Apache” score, etc.

The term “related marker” as used herein refers to one or more fragments of a particular marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆, and are also known as “NT-proBNP.” Additionally, related markers may be the result of covalent modification of the parent marker, for example by oxidation of methionine residues, ubiquitination, cysteinylation, nitrosylation (e.g., containing nitrotyrosine residues), halogenation (e.g., containing chlorotyrosine and/or bromotyrosine residues), glycosylation, complex formation, differential splicing, etc.

The sequence of the 108 amino acid BNP precursor pro-BNP (BNP₁₋₁₀₈) is as follows, with mature BNP (BNP₇₇₋₁₀₈) underlined:

(SEQ ID NO: 1)

| | |
|---|---|
| HPLGSPGSAS DLETSGLQEQ RNHLQGKLSE LQVEQTSLEP | 50 |
| LQESPRPTGV | |
| WKSREVATEG IRGHRKMVLY TLRAPRSPKM VQGSGCFGRK | 100 |
| MDRISSSSGL | |
| GCKVLRRH | 108. |

BNP₁₋₁₀₈ is synthesized as a larger precursor pre-pro-BNP having the following sequence (with the “pre” sequence shown in bold):

(SEQ ID NO: 2)

| | |
|---|---|
| MDPQTAPSRA LLLLLFLHLA FLGGRSHPLG SPGSASDLET | 50 |
| SGLQEQRNHL | |
| QGKLSELQVE QTSLEPLQES PRPTGVWKSR EVATEGIRGH | 100 |
| RKMVLYTLRA | |
| PRSPKMVQGS GCFGRKMDRI SSSSGLGCKV LRRH | 134. |

While mature BNP itself may be used as a marker in the present invention, the prepro-BNP, BNP₁₋₁₀₈ and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. patent Ser. No. 10/419,059, filed Apr. 17, 2003, which is hereby incorporated by reference in its entirety including all tables, figures and claims.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described above for BNP.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for BNP, and a significant amount (e.g., 50%) of the biologically active BNP that had been present has now been degraded into an inactive form. An immunoassay formulated with antibodies that bind a region common to the biologically active BNP and the inactive fragment(s) will overestimate the amount of biologically active BNP present in the sample by 2-fold, potentially resulting in a “false positive” result. Overestimation of the biologically active form(s) present in a sample may also have serious consequences for patient management. Considering the BNP example again, the BNP concentration may be used to determine if therapy is effective (e.g., by monitoring BNP to see if an elevated level is returning to normal upon treatment). The same “false positive” BNP result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of troponin may not detect all of the cardiac-specific isoform of interest.

Preferred assays are “configured to detect” a particular marker. That an assay is “configured to detect” a marker means that an assay can generate a detectable signal indicative of the presence or amount of a physiologically relevant concentration of a particular marker of interest. Such an assay may, but need not, specifically detect a particular marker (i.e., detect a marker but not some or all related markers). Because an antibody epitope is on the order of 8 amino acids, an immunoassay will detect other polypeptides (e.g., related markers) so long as the other polypeptides contain the epitope(s) necessary to bind to the antibody used in the assay. Such other polypeptides are referred to as being “immunologically detectable” in the assay, and would include various isoforms (e.g., splice variants). In the case of a sandwich immunoassay, related markers must contain at least the two epitopes bound by the antibody used in the assay in order to be detected. Taking BNP₇₉₋₁₀₈ as an example, an assay configured to detect this marker may also detect BNP₇₇₋₁₀₈ or BNP₁₋₁₀₈, as such molecules may also contain the epitope(s) present on BNP₇₉₋₁₀₈ to which the assay antibody binds. However, such assays may also be configured to be “sensitive” to loss of a particular epitiope, e.g., at the amino and/or carboxyl terminus of a particular polypeptide of interest as described in US2005/0148024, which is hereby incorporated by reference in its entirety. As described therein, an antibody may be selected that would bind to the amino terminus of BNP₇₉₋₁₀₈ such that it does not bind to BNP₇₇₋₁₀₈. Similar assays that bind BNP₃₋₁₀₈ and that are “sensitive” to loss of a particular epitiope, e.g., at the amino and/or carboxyl terminus are also described therein.

Preferably, the methods described hereinafter utilize one or more markers that are derived from the subject. The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, but rather that are expressed by pathogenic or infectious organisms that are correlated with a particular disease. Such markers are preferably protein, polypeptide, phospholipid, nucleic acid, prion, or small molecule markers that identify the infectious diseases described above.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care for a disease or condition. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing a future stroke in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the onset of delayed neurologic deficits in a patient after stroke, or the chance of future stroke.

The term “correlating” or “relating” as used herein in reference to the use of markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome using ROC curves.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “specifically binds” is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J Immunoassay 12: 425-43, 1991; Nelson and Griswold, Comput. Methods Programs Biomed. 27: 65-8, 1988.

Identification of Marker Panels

In accordance with the present invention, there are provided methods and systems for the identification of one or more markers useful in diagnosis, prognosis, and/or determining an appropriate therapeutic course. Suitable methods for identifying markers useful for such purposes are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims.

One skilled in the art will also recognize that univariate analysis of markers can be performed and the data from the univariate analyses of multiple markers can be combined to form panels of markers to differentiate different disease conditions. Such methods include multiple linear regression, determining interaction terms, stepwise regression, etc.

In developing a panel of markers, data for a number of potential markers may be obtained from a group of subjects by testing for the presence or level of certain markers. The group of subjects is divided into two sets. The first set includes subjects who have been confirmed as having a disease, outcome, or, more generally, being in a first condition state. For example, this first set of patients may be those diagnosed with SIRS, sepsis, severe sepsis, septic shock and/or MODS that died as a result of that disease. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or patients suffering from another cause of SIRS, and/or that lived to a particular endpoint of interest.

The data obtained from subjects in these sets preferably includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a single marker often is incapable of definitively identifying a subject as falling within a first or second group in a prospective fashion. For example, if a patient is measured as having a marker level that falls within an overlapping region in the distribution of diseased and non-diseased subjects, the results of the test may be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject (j) is expressed as:
Rⱼ=ΣwᵢI_{i,j,}
where i is the marker index, j is the subject index, wᵢ is the weighting coefficient for marker i, I is the indicator value to which the marker level for marker i is mapped for subject j, and Σ is the summation over all candidate markers i. This panel response value may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

To allow a determination of test accuracy, a “gold standard” test criterion may be selected which allows selection of subjects into two or more groups for comparison by the foregoing methods. In the case of sepsis, this gold standard may be recovery of organisms from culture of blood, urine, pleural fluid, cerebrospinal fluid, peritoneal fluid, synnovial fluid, sputum, or other tissue specimens. This implies that those negative for the gold standard are free of sepsis; however, as discussed above, 50% or more of patients exhibiting strong clinical evidence of sepsis are negative on culture. In this case, those patients showing clinical evidence of sepsis but a negative gold standard result may be omitted from the comparison groups. Alternatively, an initial comparison of confirmed sepsis subjects may be compared to normal healthy control subjects. In the case of a prognosis, mortality is a common test criterion.

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures:

at least 75% sensitivity, combined with at least 75% specificity;

ROC curve area of at least 0.6, more preferably 0.7, still more preferably at least 0.8, even more preferably at least 0.9, and most preferably at least 0.95; and/or

a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

Markers

Adiponectin

Adiponcetin (human precursor: Swiss-Prot Q15848) is a negative regulator of inflammatory and hematopoietic responses. Decreased plasma levels are also related to obesity, insulin resistance, and type II diabetes.

Alanine Aminotransferase (Serum Glutamic Pyruvic Transaminase)

Alanine aminotransferase (human precursor: Swiss-Prot P24298) is an enzyme that is expressed in the liver and heart, and so may be released into blood when the liver or heart are damaged. It is involved in cellular nitrogen metabolism and hepatic gluconeogenesis.

BNP₃₋₁₀₈ and BNP₇₉₋₁₀₈

B-type natriuretic peptide (human precursor: Swiss-Prot P16860) is a cardiac hormone having a variety of biological actions including natriuresis, diuresis, vasorelaxation, and inhibition of renin and aldosterone secretion. It is synthesized as a 134-residue precursor that is cleaved to a 108-residue proBNP molecule. This proBNP molecule is further cleaved to produce the 32-residue mature BNP molecule.

Circulating BNP-related peptides, in which the first two residues have been removed from the N-terminus of proBNP and mature BNP, have been reported. See, e.g., US2005/0148024. Preferred assays are “specific for degradation of the N-terminus.” Such a “specific” assay is configured to provide a signal that is at least 5-fold, and most preferably 10-fold or more, greater when measuring BNP₃₋₁₀₈ (or BNP₇₉₋₁₀₈) compared to an equimolar amount of BNP₁₋₁₀₈ (or BNP₇₇₋₁₀₈).

PASP

Carboxypeptidase B (human precursor: Swiss-Prot P15086) is a secreted pancreatic enzyme which catalyzes the release of C-terminal lysine and arginine residues from target proteins. PASP is secreted as a zymogen (procarboxypeptidase B), which is activated by removal of a 95 residue activation peptide. Both the active form and the activation peptide are described as being markers for severity in acute pancreatitis. PASP assays may detect one or more of procarboxypeptidase B but not active carboxypeptidase B, and activation peptide. Preferred PASP assays detect procarboxypeptidase B but not active carboxypeptidase B, active carboxypeptidase B but not procarboxypeptidase B, or both pro and active forms.

CCL4

Small inducible cytokine A4 (human: Swiss-Prot P13236), also known as Macrophage inflammatory protein 1β, is a member of the C—C motif family of chemokines. CCL4 exists as both a homodimer and a processed form MIP-1β(3-69) that forms a heterodimer with MIP-1α (4-69), and is reported to bind to CCR5 and to CCR8.

CCL16

Small inducible cytokine A16 (human: Swiss-Prot O15467) is a member of the C—C motif family of chemokines. CCL16, which is induced by IL-10, shows chemotactic activity for lymphocytes and monocytes, and potent myelosuppressive activity.

CXCL5

Small inducible cytokine B5 (human precursor: Swiss-Prot P42830), also known as ENA-78, is a member of the intercrine alpha (chemokine CxC) family. N-terminal processed forms ENA-78 (8-78) and ENA-78 (9-78) are produced by proteolytic cleavage after secretion from peripheral blood monocytes.

CXCL6

Small inducible cytokine B6 (human precursor: Swiss-Prot P80162), also known as granulocyte chemotactic protein GCP-2, is a member of the intercrine alpha (chemokine C×C) family. N-terminal processed forms containing residues 40-114, 43-114, and 46-114 of the precursor have been described.

CXCL9

Small inducible cytokine B9 (human precursor: Swiss-Prot Q07325), also known as γ-interferon induced monokine or MIG, is a member of the intercrine alpha (chemokine C×C) family.

sDR6 (Soluble DR6)

Tumor necrosis factor receptor superfamily member 21 (human precursor: Swiss-Prot O75509), also known as DR6, is a type I membrane protein related to apoptosis. Soluble circulating forms containing extracellular domain sequences may be measured.

GSTA

Glutathione-5-transferase alpha (GSTA1 human: Swiss-Prot P08263; GSTA2 human: Swiss-Prot P09210; GSTA3 human: Swiss-Prot Q16772; GSTA4 human: Swiss-Prot 015217) refers to a family of proteins that catalyze the transfer of glutathione to a protein target. GSTA1 and GSTA2 exist as homodimers or as heterodimers of GSTA1 and GSTA2. Other isoforms exist as homodimers. An assay for GSTA as that term is used herein refers to an assay that detects one or more members of the glutathione-S-transferase alpha family. Preferred assays are configured, for example, with antibodies raised against GSTA1. Such an assay could be expected to bind to circulating forms of GSTA in addition to the GSTA1 homodimer, including the GSTA2 homodimer and GSTA 1/GSTA2 heterodimer.

I-FABP

Intestinal fatty acid-binding protein (human: Swiss-Prot P12104) is believed involved in triglyceride-rich lipoprotein synthesis. I-FABP binds saturated long-chain fatty acids with a high affinity, and to unsaturated long-chain fatty acids with a lower affinity. I-FABP may also help maintain energy homeostasis by functioning as a lipid sensor. It has been reported as a marker of intestinal ischemia. See, e.g., U.S. Pat. No. 5,225,329.

L-FABP

Liver fatty acid-binding protein (human: Swiss-Prot P82289) is believed involved in straight-chain and branched-chain fatty acid metabolism. See, e.g., Atshaves et al., J. Biol. Chem. 279: 30954-65, 2004.

NGAL

Neutrophil gelatinase-associated lipocalin (human precursor Swiss-Prot P80188) is a member of the lipocalin family that forms a heterodimer with MMP-9. NGAL has been reported to be released into the circulation due to inflammatory activation of leukocytes, and as an early marker of renal injury. See, e.g., WO2005/121788.

PGRP-S

Peptidoglycan recognition protein (human precursor Swiss-Prot O75594) is a secreted protein involved in innate immunity. PGRP-S binds to bacterial peptidoglycan (a layer in the bacterial cell wall formed from linear chains of alternating N-acetyl glucosamine and N-acetyl muramic acid residues, in which each N-acetyl muramic acid group is attached to a short (4 to 5 residue) amino acid chain, normally containing the unusual amino acids D-alanine, D-glutamic acid and mesodiaminopimelic acid).

PLGF

Placental growth factor (human precursor: Swiss-Prot P49763) is a growth factor involved in angiogenesis. It circulates as both a homodimer and as a heterodimer with VEGF. Preferred assays are “insensitive” with regard to PLGF-1 and PLGF-2 isoforms. An “insensitive” assay as that term is used with regard to PLGF-1 and PLGF-2 is configured to provide a signal that is within a factor of 5, more preferably within a factor of two, and most preferably within 20%, when comparing assay results for equimolar amounts of PLGF-1 and PLGF-2. Other preferred assays are “specific for” PLGF-1 or PLGF-2 isoform, relative to the other isoform. Such a “specific” assay is configured to provide a signal that is at least 5-fold, and most preferably 10-fold or more, greater when measuring the intended PLGF isoform in comparison to equimolar amounts of the other PLGF isoform.

Protein C

Protein C (human precursor: Swiss-Prot P04070) is a vitamin K-dependent serine protease involved in blood coagulation. Synthesized as a single chain precursor, protein C is cleaved into a light chain and a heavy chain connected by a disulfide bond. The latent form of the enzyme is then activated by thrombin, which cleaves a peptide from the amino terminus. Preferred assays are “specific for activated protein C,” relative to its latent form. Such a “specific” assay is configured to provide a signal that is at least 5-fold, and most preferably 10-fold or more, greater when measuring activated protein C compared to an equimolar amount of latent protein C. Other preferred assays are specific for the latent form, such that the assay is configured to provide a signal that is at least 5-fold, and most preferably 10-fold or more, greater when measuring latent protein C compared to an equimolar amount of the active form of protein C. Still other preferred assays detect both active and latent protein C, such that the assay is configured to provide a signal that is within a factor of 5, more preferably within a factor of two, and most preferably within 20%, when measuring equimolar amounts of latent and active protein C.

IL2sRA (IL-2 Soluble Receptor Alpha)

IL-2 receptor alpha subunit (human precursor: Swiss-Prot P01589) is a type I membrane protein that binds interleukin-2. The membrane-bound receptor is a heterodimer formed with a beta chain. Soluble circulating forms containing extracellular domain sequences may be measured.

LIGHT

Tumor necrosis factor ligand superfamily member 14 (human: Swiss-Prot 043557) cytokine that binds to TNFRSF3 and activates NFKB and stimulates the proliferation of T cells. Both a type-II membrane protein form (Swiss-Prot O43557-1) and a soluble form (Swiss-Prot O43557-2) have been described.

MMP7

Matrix metalloproteinase-7 (human precursor: Swiss-Prot P09237) is a metal-binding proteolytic enzyme that hydrolyzes casein, gelatins I, III, IV, and V, and fibronectin, and activates procollagenase. Like many MMPs, MMP7 is secreted as an inactive “latent” proprotein that is activated by cleavage of an activation peptide. MMP7 differs from most MMP family members in that it lacks a conserved C-terminal protein domain.

Sphingosine Kinase I

Sphingosine kinase I (human: Swiss-Prot Q9NYA1) catalyzes the phosphorylation of sphingosine to form the lipid mediator sphingosine 1-phosphate. It binds to the calcium-binding protein calmodulin.

sTREM-1 (Soluble TREM-1)

Triggering receptor expressed on myeloid cells 1 (human precursor: Swiss-Prot Q9NP99) is a type I membrane protein related to the inflammatory response to bacterial and fungal infections. Soluble circulating forms containing extracellular domain sequences may be measured.

TREM-1sv (TREM-1 Soluble Variant)

A soluble variant of the triggering receptor expressed on myeloid cells 1 (human precursor: Swiss-Prot Q9NP99-2), TREM-1sv is detectable in biological samples.

sTNFRSF3 (Soluble TNFRSF3)

Tumor necrosis factor receptor superfamily member 3 (human precursor: Swiss-Prot P36941) is a type-I membrane protein that acts as a receptor for the heterotrimeric lymphotoxin containing LTA and LTB, and for TNFS14/LIGHT. Soluble circulating forms containing extracellular domain sequences may be measured.

sTNFRSF7 (Soluble TNFRSF7)

Tumor necrosis factor receptor superfamily member 7 (human precursor: Swiss-Prot P26842), also known as CD27 or CD27 ligand receptor, is a type-I membrane protein that acts as a receptor for Receptor for TNFSF7/CD27L. Soluble circulating forms containing extracellular domain sequences may be measured.

sTNFRSF11A (Soluble TNFRSF11A)

Tumor necrosis factor receptor superfamily member 11A (human precursor: Swiss-Prot Q9Y6Q6) also known as RANK, is a type-I membrane protein that acts as a receptor for TNFSF11/RANKL/TRANCE/OPGL. RANK interacts with TRAF1, TRAF2, TRAF3, TRAF5 and TRAF6. Soluble circulating forms containing extracellular domain sequences may be measured.

TNF-sR14 (Soluble TNFRSF14)

Tumor necrosis factor receptor superfamily member 14 (human precursor: Q92956) is a type-I membrane protein that acts as a receptor for TNFSF14 (LIGHT), and is involved in lymphocyte activation. Soluble circulating forms containing extracellular domain sequences may be measured.

UCRP

Ubiquitin cross-reactive protein (human precursor: Swiss-Prot P05161), also known as Interferon-induced 17 kDa protein, is conjugated to certain target proteins in a manner similar to ubiquitin, although via a separate enzymatic pathway. Targets include SERPINA3G, JAK1, MAPK3, and PLCG1. A C-terminal octapeptide is removed to provide a mature 15 kDa form.

uPAR

Urokinase plasminogen activator surface receptor (human precursor: Swiss-Prot Q03405) is a GPI-anchored membrane protein that is a receptor for urokinase plasminogen activator. A secreted splice variant also has been described.

A panel consisting of the markers referenced herein and/or their related markers may be constructed to provide relevant information related to the diagnosis of interest. Such a panel may be constructed using 1, 2, 3, 4, 5, 6, 7, 8, 9, 10, 11, 12, 13, 14, 15, 16, 17, 18, 19, 20, or more individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity.

The following table provides a list of additional preferred markers for use in the present invention. Further detail is provided in US2005/0148029, which is hereby incorporated by reference in its entirety. As described herein, markers related to each of these markers are also encompassed by the present invention.

| Marker | Classification |
|---|---|
| Myoglobin | Tissue injury |
| E-selectin | Tissue injury |
| VEGF | Tissue injury |
| EG-VEGF | Tissue injury |
| Troponin I and complexes | Myocardial injury |
| Troponin T and complexes | Myocardial injury |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| ANP | Blood pressure regulation |
| CNP | Blood pressure regulation |
| Kininogen | Blood pressure regulation |
| CGRP II | Blood pressure regulation |
| urotensin II | Blood pressure regulation |
| BNP | Blood pressure regulation |
| NT-proBNP | Blood pressure regulation |
| proBNP | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 (and/or angiotensinogen 1) | Blood pressure regulation |
| angiotensin 2 (and/or angiotensinogen 2) | Blood pressure regulation |
| angiotensin 3 (and/or angiotensinogen 3) | Blood pressure regulation |
| Bradykinin | Blood pressure regulation |
| Tachykinin-3 | Blood pressure regulation |
| calcitonin | Blood pressure regulation |
| Renin | Blood pressure regulation |
| Urodilatin | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Protein C | Coagulation and hemostasis |
| Soluble Endothelial Protein C Receptor | Coagulation and hemostasis |
| (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| Calponin | Vascular tissue |
| CSRP2 | Vascular tissue |
| elastin | Vascular tissue |
| Endothelial cell-selective adhesion | Vascular tissue |
| molecule (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| transgelin | Vascular tissue |
| Carboxyterminal propeptide of type I | Collagen synthesis |
| procollagen (PICP) | |
| Collagen carboxyterminal telopeptide (ICTP) | Collagen degradation |
| APRIL (TNF ligand superfamily member 13) | Inflammatory |
| CD27 (TNFRSF7) | Inflammatory |
| Complement C3a | Inflammatory |
| CCL-5 (RANTES) | Inflammatory |
| CCL-8 (MCP-2) | Inflammatory |
| CCL-16 | Inflammatory |
| CCL-19 (macrophage inflammatory | Inflammatory |
| protein-3β) | |
| CCL-20 (MIP-3α) | Inflammatory |
| CCL-23 (MIP-3) | Inflammatory |
| CXCL-5 (small inducible cytokine B5) | Inflammatory |
| CXCL-9 (small inducible cytokine B9) | Inflammatory |
| CXCL-13 (small inducible cytokine B13) | Inflammatory |
| CXCL-16 (small inducible cytokine B16) | Inflammatory |
| DPP-II (dipeptidyl peptidase II) | Inflammatory |
| DPP-IV (dipeptidyl peptidase IV) | Inflammatory |
| Glutathione S Transferase | Inflammatory |
| HIF 1 ALPHA | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Inducible nitric oxide synthase | Inflammatory |
| Intracellular adhesion molecule | Inflammatory |
| NGAL (Lipocalin-2) | Inflammatory |
| Lactate dehydrogenase | Inflammatory |
| MCP-1 | Inflammatory |
| MMP-1 | Inflammatory |
| MMP-2 | Inflammatory |
| MMP-3 | Inflammatory |
| MMP-7 | Inflammatory |
| MMP-9 | Inflammatory |
| TIMP-1 | Inflammatory |
| TIMP-2 | Inflammatory |
| TIMP-3 | Inflammatory |
| NGAL | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| TNFRSF3 (lymphotoxin β receptor) | Inflammatory |
| Transforming growth factor beta | Inflammatory |
| TREM-1 | Inflammatory |
| TREM-1sv | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| Myeloperoxidase (MPO) | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Prostaglandin D-synthase | Inflammatory |
| Prostaglandin E2 | Inflammatory |
| RANK ligand | Inflammatory |
| RANK (TNFRSF11A) | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| Neutrophil elastase | Pulmonary injury |
| KL-6 | Pulmonary injury |
| LAMP 3 | Pulmonary injury |
| LAMP3 | Pulmonary injury |
| Lung Surfactant protein A | Pulmonary injury |
| Lung Surfactant protein B | Pulmonary injury |
| Lung Surfactant protein C | Pulmonary injury |
| Lung Surfactant protein D | Pulmonary injury |
| phospholipase D | Pulmonary injury |
| PLA2G5 | Pulmonary injury |
| SFTPC | Pulmonary injury |
| MAPK10 | Neural tissue injury |
| KCNK4 | Neural tissue injury |
| KCNK9 | Neural tissue injury |
| KCNQ5 | Neural tissue injury |
| 14-3-3 | Neural tissue injury |
| 4.1B | Neural tissue injury |
| APO E4-1 | Neural tissue injury |
| myelin basic protein | Neural tissue injury |
| Atrophin 1 | Neural tissue injury |
| Brain derived neurotrophic factor | Neural tissue injury |
| Brain fatty acid binding protein | Neural tissue injury |
| Brain tubulin | Neural tissue injury |
| CACNA1A | Neural tissue injury |
| Calbindin D | Neural tissue injury |
| Calbrain | Neural tissue injury |
| Carbonic anhydrase XI | Neural tissue injury |
| CBLN1 | Neural tissue injury |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CK-BB | Neural tissue injury |
| CRHR1 | Neural tissue injury |
| C-tau | Neural tissue injury |
| DRPLA | Neural tissue injury |
| GFAP | Neural tissue injury |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIP2 | Neural tissue injury |
| LDH | Neural tissue injury |
| Myelin basic protein | Neural tissue injury |
| NCAM | Neural tissue injury |
| NT-3 | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1, 2, 3, 4 | Neural tissue injury |
| NRG2 | Neural tissue injury |
| PACE4 | Neural tissue injury |
| phosphoglycerate mutase | Neural tissue injury |
| PKC gamma | Neural tissue injury |
| proteolipid protein | Neural tissue injury |
| PTEN | Neural tissue injury |
| PTPRZ1 | Neural tissue injury |
| RGS9 | Neural tissue injury |
| RNA Binding protein Regulatory Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| SREB3 | Neural tissue injury |
| STAC | Neural tissue injury |
| STX1A | Neural tissue injury |
| STXBP1 | Neural tissue injury |
| Syntaxin | Neural tissue injury |
| thrombomodulin | Neural tissue injury |
| transthyretin | Neural tissue injury |
| adenylate kinase-1 | Neural tissue injury |
| BDNF | Neural tissue injury |
| neurokinin A | Neural tissue injury |
| neurokinin B | Neural tissue injury |
| s-acetyl Glutathione | apoptosis |
| cytochrome C | apoptosis |
| Caspase 3 | apoptosis |
| Cathepsin D | apoptosis |
| α-spectrin | apoptosis |

Protein Modification and Sepsis

Ubiquitin-mediated degradation of proteins plays an important role in the control of numerous processes, such as the way in which extracellular materials are incorporated into a cell, the movement of biochemical signals from the cell membrane, and the regulation of cellular functions such as transcriptional on-off switches. The ubiquitin system has been implicated in the immune response and development. Ubiquitin is a 76-amino acid polypeptide that is conjugated to proteins targeted for degradation. The ubiquitin-protein conjugate is recognized by a 26S proteolytic complex that splits ubiquitin from the protein, which is subsequently degraded.

It has been reported that sepsis stimulates protein breakdown in skeletal muscle by a nonlysosomal energy-dependent proteolytic pathway, and because muscle levels of ubiquitin mRNA were also increased, the results were interpreted as indicating that sepsis-induced muscle protein breakdown is caused by upregulated activity of the energy-ubiquitin-dependent proteolytic pathway. The same proteolytic pathway has been implicated in muscle breakdown caused by denervation, fasting, acidosis, cancer, and burn injury. Thus, levels of ubiquitinated proteins generally, or of specific ubiquitin-protein conjugates or fragments thereof, can be measured as additional markers of the invention. See, Tiao et al., J. Clin. Invest. 99: 163-168, 1997. Moreover, circulating levels of ubiquitin itself can be a useful marker in the methods described herein. See, e.g., Majetschak et al., Blood 101:1882-90, 2003.

Interestingly, ubiquitination of a protein or protein fragment may convert a non-specific marker into a more specific marker of sepsis. For example, muscle damage can increase the concentration of muscle proteins in circulation. But sepsis, by specifically upregulating the ubiquitination pathway, may result in an increase of ubiquitinated muscle proteins, thus distinguishing non-specific muscle damage from sepsis-induced muscle damage.

The skilled artisan will recognize that an assay for ubiquitin may be designed that recognizes ubiquitin itself, ubiquitin-protein conjugates, or both ubiquitin and ubiquitin-protein conjugates. For example, antibodies used in a sandwich immunoassay may be selected so that both the solid phase antibody and the labeled antibody recognize a portion of ubiquitin that is available for binding in both unconjugated ubiquitin and ubiquitin conjugates. Alternatively, an assay specific for ubiquitin conjugates of the muscle protein troponin could use one antibody (on a solid phase or label) that recognizes ubiquitin, and a second antibody (the other of the solid phase or label) that recognizes troponin.

The present invention contemplates measuring ubiquitin conjugates of any marker described herein and/or their related markers. Preferred ubiquitin-muscle protein conjugates for detection as markers include, but are not limited to, troponin I-ubiquitin, troponin T-ubiquitin, troponin C-ubiquitin, binary and ternary troponin complex-ubiquitin, actin-ubiquitin, myosin-ubiquitin, tropomyosin-ubiquitin, and α-actinin-ubiquitin and ubiquitinated markers related thereto.

In similar fashion, other modifications of the markers described herein, or markers related thereto, can be detected. For example, nitrotyrosine, chlorotyrosine, and/or bromotyrosine may be formed by the action of myeloperoxidase in sepsis. See, e.g., U.S. Pat. No. 6,939,716. Assays for nitrotyrosine, chlorotyrosine, and/or bromotyrosine may be designed that recognize one or more of these individual modified amino acids, one or more markers containing one or more of the modified amino acids, or both modified amino acid(s) and modified marker(s).

Exemplary SIRS Markers and Marker Panels

Exemplary markers and marker panels are preferably designed to diagnose sepsis, to differentiate sepsis, severe sepsis, septic shock and/or MODS from other causes of SIRS, to assist in the stratification of risk in sepsis patients, and most preferably to direct treatment of subjects. In addition to latent, activated, and/or total protein C, BNP₃₋₁₀₈, BNP₇₉₋₁₀₈, CCL4, CXCL6, sDR6, glutathione-S-transferase A, intestinal fatty acid binding protein, placental growth factor, IL2sRA, sphingosine kinase I, and uPAR, particularly preferred markers are matrix metalloproteinase 9 (MMP-9), interleukin-1β (IL-1β), interleukin-6 (IL-6), interleukin-8 (IL-8), interleukin-10 (IL-10), interleukin-22 (IL-22), IL-1receptor agonist (IL-1ra), CXCL6, CXCL13, CXCL16, CCL8, CCL19, CCL20, CCL23, CCL26, D-dimer, HMG-1, tumor necrosis factor-α (TNF-α), B-type natriuretic protein (BNP), A-type natriuretic protein (ANP), B-type natriuretic protein (BNP), C-reactive protein (CRP), caspase-3, calcitonin, procalcitonin₃₋₁₁₆, soluble DPP-IV, soluble FAS ligand (sFasL), creatine kinase-BB (CK-BB), vascular endothelial growth factor (VEGF), myeloperoxidase (MPO), and soluble intercellular adhesion molecule-1 (sICAM-1), or immunologically detectable related polypeptides, including fragments of these proteins or their biosynthetic precursors.

Preferred panels include one or more markers related to inflammation and one or more markers related to blood pressure regulation; one or more markers related to inflammation and one or more markers related to coagulation and hemostasis; or one or more markers related to inflammation, one or more markers related to coagulation and hemostasis, and one or more markers related to blood pressure regulation.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of markers using a single test device. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody conjugated to a solid phase and a second antibody conjugated to a signal development element. Such assay devices are configured to perform a sandwich immunoassay for one or more analytes. These assay devices will preferably further comprise a sample application zone, and a flow path from the sample application zone to a second device region comprising the first antibody conjugated to a solid phase.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001, which is hereby incorporated by reference in its entirety.

A panel consisting of the markers referenced above may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses. Other measurement strategies applicable to the methods described herein include chromatography (e.g., HPLC), mass spectrometry, receptor-based assays, and combinations of the foregoing.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g., Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999. With regard to SIRS, sepsis, severe sepsis, and septic shock, recent guidelines provide additional information for the clinician. See, e.g., Dellinger et al., Crit. Care Med. 32: 858-73, 2004, which is hereby incorporated by reference in its entirety.

While the present invention may be used to determine if any SIRS-related (that is, applicable to SIRS, sepsis, severe sepsis, septic shock, and MODS) treatment should be undertaken at all, the invention is preferably used to assign a particular treatment regimen from amongst two or more possible choices of SIRS-related treatment regimens. For example, in exemplary embodiments, the present invention is used to determine if subjects should receive standard therapy or early goal-directed therapy. Thus, the methods and compositions described herein may be used to select one or more of the following treatments for inclusion in a therapy regimen:

Administration of intravenous antibiotic therapy;

maintenance of a central venous pressure of 8-12 mm Hg;

administration of crystalloids and/or colloids, preferably to maintain such a central venous pressure;

maintenance of a mean arterial pressure of >65 mm Hg;

administration of one or more vasopressors (e.g., norepinephrine, dopamine, and/or vasopressin) and/or vasodilators (e.g., prostacyclin, pentoxifylline, N-acetyl-cysteine);

administration of one or more corticosteroids (e.g., hydrocortisone);

administration of recombinant activated protein C;

maintenance of a central venous oxygen saturation of ≧70%;

administration of transfused red blood cells to a hematocrit of at least 30%;

administration of one or more inotropics (e.g., dobutamine); and

administration of mechanical ventilation.

This list is not meant to be limiting. In addition, since the methods and compositions described herein provide prognostic information, the panels and markers of the present invention may be used to monitor a course of treatment. For example, improved or worsened prognostic state may indicate that a particular treatment is or is not efficacious.

### EXAMPLES

The following examples serve to illustrate the present invention. These examples are in no way intended to limit the scope of the invention.

### Example 1

### Subject Population and Sample Collection

Test subjects in disease categories were enrolled as part of a prospective sepsis study conducted by Biosite Incorporated at 10 clinical sites in the United States. Enrollment criteria were: age 18 or older and presenting with two or more SIRS criteria, and confirmed or suspected infection and/or lactate levels greater than 2.5 mmol/L. Exclusion criteria were: pregnancy, cardiac arrest, and patients under Do Not Resuscitate (DNR) orders. Samples were collected by trained personnel in standard blood collection tubes with EDTA as the anticoagulant. The plasma was separated from the cells by centrifugation, frozen, and stored at −20° C. or colder until analysis. The plasma was frozen within 1 hour. Clinical histories are available for each of the patients to aid in the statistical analysis of the assay data. Patients were assigned a final diagnosis by a physician at the clinical site using the standard medical criteria in use at each clinical site. Patients were diagnosed as having systemic inflammatory response syndrome (SIRS), sepsis, severe sepsis, septic shock or multiple organ dysfunction syndrome (MODS).

Samples from apparently healthy blood donors were purchased from Golden West Golden West Biologicals, Inc., Temecula, Calif., and were collected according to a defined protocol. Samples were collected from normal healthy individuals with no current clinical suspicion or evidence of disease. Blood was collected by trained personnel in standard blood collection tubes with EDTA as the anticoagulant. The plasma was separated from the cells by centrifugation, frozen, and stored at −20° C. or colder until analysis.

### Example 2

### Biochemical Analyses

Analytes (e.g., markers and/or polypeptides related thereto) were measured using standard immunoassay techniques. These techniques involve the use of antibodies to specifically bind the analyte(s) of interest. Immunoassays were performed using TECAN Genesis RSP 200/8 or Perkin Elmer Minitrak Workstations, or using microfluidic devices manufactured at Biosite Incorporated essentially as described in WO98/43739, WO98/08606, WO98/21563, and WO93/24231. Analytes may be measured using a sandwich immunoassay or using a competitive immunoassay as appropriate, depending on the characteristics and concentration range of the analyte of interest. For analysis, an aliquot of plasma was thawed and samples analyzed as described below. Activated Protein C has benzamidine added to a final concentration of 2 mM.

The assays were calibrated using purified proteins (that is either the same as or related to the selected analyte, and that can be detected in the assay) diluted gravimetrically into EDTA plasma treated in the same manner as the sample population specimens. Endogenous levels of the analyte present in the plasma prior to addition of the purified marker protein was measured and taken into account in assigning the marker values in the calibrators. When necessary to reduce endogenous levels in the calibrators, the endogenous analyte was stripped from the plasma using standard immunoaffinity methods. Calibrators were assayed in the same manner as the sample population specimens, and the resulting data used to construct a “dose-response” curve (assay signal as a function of analyte concentration), which may be used to determine analyte concentrations from assay signals obtained from subject specimens.

Individual assays were configured to bind the following markers, and results are reported in the following examples using the following units: adiponectin—ng/mL; adrenomedullin—pg/mL; angiotensinogen—μg/mL; apolipoprotein C1—ng/mL; Big ET-1—pg/mL; BNP—pg/mL; BNP₁₋₁₀₈—pg/mL; BNP₃₋₁₀₈—pg/mL; BNP₇₉₋₁₀₈—pg/mL; calcitonin—pg/mL; caspase-3—ng/mL; CCL4—pg/mL; CCL5—ng/mL; CCL8—ng/mL; CCL16—ng/mL; CCL19—ng/mL; CCL20—pg/mL; CCL23—ng/mL; CCL26—pg/mL; CK-BB—ng/mL; CK-MB—ng/mL; CRP—μg/mL; CXCL5—pg/mL; CXCL6—pg/mL; CXCL9—ng/mL; CXCL13—pg/mL; CXCL16—ng/mL; complement C3A—ng/mL; cystatin C—ng/mL; D-dimer—ng/mL; sDR6—ng/mL; sFasL—ng/mL; glutathione-S-transferase A—ng/mL; HSP-60—ng/mL; HMG-1—ng/mL; sICAM-1—ng/mL; I-FABP—ng/mL; IGFBP-1—ng/mL; IL2sRA—ng/mL; IL-10—pg/mL; IL-1β—pg/mL; IL-1ra—pg/mL; IL-6—pg/mL; IL-8—pg/mL; IL-22—pg/mL; MCP1—pg/mL; MIF—pg/mL; MMP-9—ng/mL; MPO—ng/mL; protein C (activated or total activated +latent)—ng/mL; myoglobin—ng/mL; NGAL—ng/mL; PAI-1—pg/mL; PLGF—pg/mL; Pten—ng/mL; pulmonary surfactant protein A—ng/mL; pulmonary surfactant protein B—ng/mL; pulmonary surfactant protein D—ng/mL; RAGE—ng/mL; sphingosine kinase 1—ng/mL; TIMP-1—μg/mL; TNF-α—pg/mL; TNFR1a—pg/mL; sTNFRSF3—ng/ml; sTNFRSF7—ng/mL; sTNFRSF11A—ng/mL; sTNFRSF14—pg/mL; sTREM-1—ng/mL; TREM-1sv—ng/mL; tissue factor—pg/mL; UCRP—ng/mL; uPAR—ng/mL; and VCAM-1—ng/mL.

### Example 3

### Microtiter Plate-Based Biochemical Analyses

For the sandwich immunoassay in microtiter plates, a monoclonal antibody directed against a selected analyte was biotinylated using N-hydroxysuccinimide biotin (NHS-biotin) at a ratio of about 5 NHS-biotin moieties per antibody. The antibody-biotin conjugate was then added to wells of a standard avidin 384 well microtiter plate, and antibody conjugate not bound to the plate was removed. This formed the “anti-marker” in the microtiter plate. Another monoclonal antibody directed against the same analyte was conjugated to alkaline phosphatase, for example using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Biotinylated antibodies were pipetted into microtiter plate wells previously coated with avidin and incubated for 60 min. The solution containing unbound antibody was removed, and the wells washed with a wash buffer, consisting of 20 mM borate (pH 7.42) containing 150 mM NaCl, 0.1% sodium azide, and 0.02% Tween-20. The plasma samples (10 μL, or 20 μL for CCL4) containing added HAMA inhibitors were pipetted into the microtiter plate wells, and incubated for 60 min. The sample was then removed and the wells washed with a wash buffer. The antibody-alkaline phosphatase conjugate was then added to the wells and incubated for an additional 60 min, after which time, the antibody conjugate was removed and the wells washed with a wash buffer. A substrate, (AttoPhos®, Promega, Madison, Wis.) was added to the wells, and the rate of formation of the fluorescent product is related to the concentration of the analyte in the sample tested.

For competitive immunoassays in microtiter plates, a murine monoclonal antibody directed against a selected analyte was added to the wells of a microtiter plate and immobilized by binding to goat anti-mouse antibody that is pre-absorbed to the surface of the microtiter plate wells (Pierce, Rockford, Ill.). Any unbound murine monoclonal antibody was removed after a 60 minute incubation. This forms the “anti-marker” in the microtiter plate. A purified polypeptide that is either the same as or related to the selected analyte, and that can be bound by the monoclonal antibody, was biotinylated as described above for the biotinylation of antibodies. This biotinylated polypeptide was mixed with the sample in the presence of HAMA inhibitors, forming a mixture containing both exogenously added biotinylated polypeptide and any unlabeled analyte molecules endogenous to the sample. The amount of the monoclonal antibody and biotinylated marker added depends on various factors and was titrated empirically to obtain a satisfactory dose-response curve for the selected analyte.

This mixture was added to the microtiter plate and allowed to react with the murine monoclonal antibody for 120 minutes. After the 120 minute incubation, the unbound material was removed, and Neutralite-Alkaline Phosphatase (Southern Biotechnology; Birmingham, Ala.) was added to bind to any immobilized biotinylated polypeptide. Substrate (as described above) was added to the wells, and the rate of formation of the fluorescent product was related to the amount of biotinylated polypeptide bound, and therefore was inversely related to the endogenous amount of the analyte in the specimen.

### Example 4

### Microfluidic Device-Based Biochemical Analyses

Immunoassays were performed using microfluidic devices essentially as described in Chapter 41, entitled “Near Patient Tests: Triage® Cardiac System,” in The Immunoassay Handbook, 2^{nd} ed., David Wild, ed., Nature Publishing Group, 2001.

For sandwich immunoassays, a plasma sample is added to the microfluidic device that contains all the necessary assay reagents, including HAMA inhibitors, in dried form. The plasma passes through a filter to remove particulate matter. Plasma enters a “reaction chamber” by capillary action. This reaction chamber contains fluorescent latex particle-antibody conjugates (hereafter called FETL-antibody conjugates) appropriate to an analyte of interest, and may contain FETL-antibody conjugates to several selected analytes. The FETL-antibody conjugates dissolve into the plasma to form a reaction mixture, which is held in the reaction chamber for an incubation period (about a minute) to allow the analyte(s) of interest in the plasma to bind to the antibodies. After the incubation period, the reaction mixture moves down the detection lane by capillary action. Antibodies to the analyte(s) of interest are immobilized in discrete capture zones on the surface of a “detection lane.” Analyte/antibody-FETL complexes formed in the reaction chamber are captured on an appropriate detection zone to form a sandwich complex, while unbound FETL-antibody conjugates are washed from the detection lane into a waste chamber by excess plasma. The amount of analyte/antibody-FETL complex bound on a capture zone is quantified with a fluorometer (Triage® MeterPlus, Biosite Incorporated) and is related to the amount of the selected analyte in the plasma specimen.

For competitive immunoassays, the procedure and process is similar to that described for sandwich immunoassays, with the following exceptions. In one configuration, fluorescent latex particle-marker (FETL-marker) conjugates are provided in the reaction chamber, and are dissolved in the plasma to form a reaction mixture. This reaction mixture contains both the unlabeled analyte endogenous to the sample, and the FETL-marker conjugates. When the reaction mixture contacts the capture zone for a analyte of interest, the unlabeled endogenous analyte and the FETL-marker conjugates compete for the limited number of antibody binding sites. Thus, the amount of FETL-marker conjugate bound to the capture zone is inversely related to the amount of analyte endogenously present in the plasma specimen. In another configuration, antibody-FETL conjugates are provided in the reaction chamber as described above for sandwich assays. In this configuration, the capture zone contains immobilized marker on the surface of the detection lane. Free antibody-FETL conjugates bind to this immobilized marker on the capture zone, while antibody-FETL conjugates bound to an analyte of interest do not bind as readily or at all to this immobilized marker. Again, the amount of FETL captured in the zone is inversely related to the amount of the selected analyte in the plasma specimen. One skilled in the art will recognize that either configuration may be used depending on the characteristics and concentrations of the selected analyte(s).

### Example 5

### Marker Panels

Using the methods described in PCT application no. US03/41426, filed Dec. 23, 2003, exemplary panels for diagnosis and risk stratification in SIRS are identified. Starting with a large number of potential markers, an iterative procedure is applied. In this procedure, individual threshold concentrations for the markers are not used as cutoffs per se, but are used as values to which the assay values for each patient are compared and normalized. Rather, a “window” of assay values between a minimum and maximum marker concentration (calculated as midpoint±midpoint×linear range in the tables below) is determined. Measured marker concentrations above the maximum are assigned a value of 1 and measured marker concentrations below the minimum are assigned a value of 0; measured marker concentrations within the window are linearly interpolated to a value of between 0 and 1. The value is then multiplied by a weighting factor (weight average in the tables below). The absolute values of the weights for all of the individual markers add up to 1. A negative weight for a marker implies that the assay values for the control group are higher than those for the diseased group. A “panel response” is calculated using the midpoint, linear range “window,” and weighting factors. The panel responses for the entire population of “disease group” and “controls” are subjected to ROC and/or correlation analysis, and a panel response cutoff is selected to yield the desired sensitivity and specificity for separating the “disease” and “non-disease” populations. After each set of iterations, the weakest contributors to the equation may be eliminated and the iterative process started again with the reduced number of markers. This process is continued until a minimum number of markers that will still result in acceptable sensitivity and specificity of the panel is obtained.

Using these methods, various panels may be defined, depending upon the identity of the markers selected, the number of markers for the final panel, and the selection of “disease” and “non-disease” populations for performing the optimization. Average ROC areas, sensitivities, and specificities calculated from 100 separate calculated “anneals” are used to determine the particular panel parameters.

Diagnostic and/or prognostic panels can be defined using a number of different marker combinations. Depending on the selection of “diseased” and “nondiseased” populations, the resulting panels can provide additional prognostic information, depending upon the treatment regimen. As described herein, the average ROC area provides an indication of how well the two groups under study may be discriminated using the particular panel (defined by the markers and their associated parameters). A plurality of panel response thresholds can be calculated from the same panel (or from different subsets of markers in the same panel), each threshold providing different information. For example, as SIRS, sepsis, severe sepsis, septic shock, and MODS represent different, but related, clinical states, individual thresholds can be established to provide diagnostic and prognostic information for one or more clinical states. Alternatively, one threshold can provide prognostic information, another threshold can provide diagnostic information, and/or another threshold can provide treatment assignment.

### Example 6

### Use of Individual Markers

In addition to their use in panels, the various markers described herein may also be used individually to provide prognostic and diagnostic information. The following tables provide statistics from measurements of individual markers in patients diagnosed as having systemic inflammatory response syndrome (SIRS), sepsis, severe sepsis, septic shock or multiple organ dysfunction syndrome (MODS), and in normal controls. Samples measured in patients were “first draws” obtained upon enrollment in the study described in Example 1.

TABLE 1

| | | | | Severe | Septic | | | | | Severe | Septic | |
|---|---|---|---|---|---|---|---|---|---|---|---|---|
| | Normal | SIRS | Sepsis | Sepsis | Shock | MODS | Normal | SIRS | Sepsis | Sepsis | Shock | MODS |
| | N | | | | | | Concentration (median) | | | | | |
| Adiponectin | 277 | 20 | 58 | 15 | 13 | — | 2409 | 5031 | 2890 | 3607 | 3025 | — |
| Adrenomedullin | 274 | 90 | 168 | 29 | 19 | 13 | 75.5 | 285.0 | 376.2 | 667.1 | 587.1 | 1532.6 |
| Angiotensinogen | 273 | 40 | 85 | 23 | 12 | 9 | 58.9 | 65.5 | 50.5 | 83.7 | 63.6 | 69.5 |
| Apolipoprotein C1 | 277 | 14 | 38 | 18 | 8 | — | 1655 | 981 | 970 | 798 | 898 | — |
| Big Endothelin-1 | 277 | 74 | 126 | 25 | 17 | 12 | <13 | 23 | 29 | 66.4 | 68.6 | 70.3 |
| BNP₁₋₁₀₈ | 0 | 20 | 32 | 8 | 7 | 6 | — | 102.1 | 129.4 | 129.0 | 236.0 | 379.2 |
| BNP₇₉₋₁₀₈ | 273 | 20 | 32 | 8 | 7 | 6 | 0.8 | 7.7 | 4.8 | 5.4 | 10.0 | 12.9 |
| BNP (BNP₇₇₋₁₀₈) | 252 | 120 | 197 | 32 | 16 | 20 | 14.8 | 5.3 | 43.3 | 60.3 | 191.8 | 307.7 |
| BNP₃₋₁₀₈ | 278 | 116 | 184 | 32 | 15 | 19 | 34.9 | 63.4 | 183.1 | 333.6 | 706.9 | 559.3 |
| Complement C3a | 0 | 53 | 98 | 23 | 14 | 11 | — | 775.7 | 912.4 | 696.3 | 746.2 | 717.4 |
| Calcitonin | 277 | 114 | 191 | 32 | 15 | 19 | 10.8 | 3.7 | 7.0 | 2.3 | 17.9 | 2.1 |
| Caspase-3 | 279 | 112 | 196 | 30 | 17 | 17 | 0.8 | 5.7 | 7.3 | 7.7 | 6.8 | 6.0 |
| CCL16 | 277 | 7 | 16 | 5 | 4 | — | 13 | 6 | 9 | 7 | 14 | — |
| CCL19 | 275 | 115 | 193 | 31 | 16 | 19 | 0.2 | 0.5 | 0.7 | 0.8 | 1.1 | 0.4 |
| CCL20 | 274 | 105 | 182 | 31 | 19 | 14 | 6.5 | 49.2 | 82.6 | 275.8 | 317.9 | 346.4 |
| CCL23 | 279 | 110 | 194 | 30 | 16 | 17 | 0.1 | 0.5 | 0.8 | 0.7 | 1.4 | 1.0 |
| CCL26 | 82 | 14 | 38 | 18 | 8 | — | 26 | 23 | 30 | 30 | 24 | — |
| CCL4 (MIP1β) | 273 | 103 | 181 | 31 | 19 | 13 | 1.2 | 186.4 | 234.6 | 283.2 | 420.0 | 428.2 |
| CCL5 | 277 | 89 | 133 | 23 | 11 | 12 | 1.1 | 34.0 | 50.0 | 18.7 | 13.3 | 5.6 |
| CCL8 | 276 | 109 | 193 | 29 | 17 | 15 | 0.0 | 0.0 | 0.0 | 0.0 | 0.0 | 0.0 |
| CK-BB | 258 | 118 | 195 | 32 | 16 | 20 | 0.5 | 0.0 | 0.1 | 0.0 | 0.0 | 0.0 |
| CK-MB | 215 | 77 | 158 | 35 | 23 | — | <1 | <1 | <1 | <1 | <1 | — |
| C-reactive protein | 265 | 117 | 191 | 30 | 16 | 18 | 0.0 | 34.7 | 49.6 | 61.1 | 64.9 | 55.0 |
| (CRP) | | | | | | | | | | | | |
| CXCL5 | 277 | 14 | 38 | 18 | 8 | — | 90 | 141 | 253 | 183 | 52 | — |
| CXCL9 | 0 | 11 | 24 | 9 | 4 | — | — | 2.8 | 2.4 | 0.7 | 2.6 | — |
| CXCL13 | 278 | 110 | 192 | 30 | 17 | 16 | 2.1 | 17.4 | 92.8 | 157.4 | 209.7 | 244.2 |
| CXCL16 | 284 | 91 | 137 | 24 | 11 | 12 | 3.1 | 5.7 | 7.5 | 9.4 | 10.7 | 15.4 |
| CXCL6 | 273 | 103 | 181 | 31 | 19 | 13 | 11.8 | 75.8 | 98.9 | 99.0 | 80.2 | 93.6 |
| Cystatin C | 220 | 83 | 159 | 24 | 12 | 10 | <1000 | <1000 | <1000 | 2664.8 | 3122.2 | 3750.9 |
| D-Dimer | 248 | 119 | 200 | 32 | 16 | 20 | 76.4 | 1212.3 | 1614.7 | 3715.9 | 2083.5 | 3164.9 |
| sDR6 | 272 | 105 | 182 | 31 | 18 | 12 | 11.5 | 25.7 | 38.8 | 73.1 | 137.0 | 67.6 |
| Glutathione- | 271 | 103 | 171 | 28 | 18 | 14 | 1.2 | 2.0 | 2.5 | 1.5 | 3.8 | 4.0 |
| S-transferase A | | | | | | | | | | | | |
| (GSTA) | | | | | | | | | | | | |
| HSP-60 | 277 | 20 | 58 | 15 | 13 | — | 0.8 | 1.7 | 2.5 | 4.0 | 3.2 | — |
| HMG-1 | 277 | 111 | 194 | 33 | 16 | 19 | 1.2 | 3.3 | 3.5 | 3.0 | 3.8 | 5.1 |
| I-FABP | 273 | 22 | 33 | 9 | 7 | 6 | 1.3 | 1.4 | 0.8 | 0.9 | 1.2 | 4.3 |
| IGFBP-1 | 277 | 31 | 77 | 25 | 20 | — | 45 | 95 | 42 | 108 | 62 | — |
| IL-10 | 274 | 100 | 179 | 29 | 18 | 14 | 0.0 | 17.8 | 31.5 | 69.1 | 56.9 | 42.4 |
| IL-1β | 274 | 35 | 57 | 19 | 11 | 6 | 6.2 | 16.5 | 16.1 | 4.2 | 1.9 | 0.1 |
| IL-1ra | 256 | 120 | 200 | 32 | 16 | 20 | 210.8 | 396.1 | 590.5 | 1039.9 | 2354.4 | 2257.1 |
| IL-22 | 280 | 115 | 190 | 32 | 16 | 19 | 7.1 | 7.5 | 12.2 | 24.7 | 30.1 | 17.5 |
| IL2sRA | 274 | 80 | 152 | 29 | 16 | 13 | 0.5 | 1.0 | 1.4 | 1.9 | 3.2 | 2.3 |
| IL-6 | 281 | 113 | 192 | 32 | 15 | 19 | 0.0 | 61.5 | 222.7 | 312.1 | 251.3 | 345.7 |
| IL-8 | 263 | 119 | 200 | 32 | 16 | 20 | 0.5 | 0.0 | 0.0 | 0.0 | 0.0 | 0.0 |
| MCP-1 | 274 | 53 | 98 | 23 | 14 | 11 | 29.0 | 58.0 | 64.6 | 75.2 | 85.2 | 151.6 |
| MIF | 277 | 56 | 103 | 15 | 9 | 8 | 13 | 57 | 74 | 64.0 | 91.0 | 88.4 |
| MMP9 | 270 | 114 | 190 | 32 | 16 | 19 | 19.8 | 100.9 | 83.0 | 63.5 | 43.2 | 47.3 |
| MPO | 258 | 116 | 196 | 30 | 16 | 20 | 13.7 | 38.1 | 59.8 | 63.8 | 132.8 | 104.8 |
| MYOGLOBIN | 264 | 118 | 198 | 32 | 16 | 20 | 71.6 | 107.4 | 133.5 | 250.4 | 385.3 | 433.6 |
| NGAL | 221 | 83 | 161 | 35 | 23 | — | 307 | 1000 | 1000 | 1000 | 1000 | — |
| PAI-1 | 278 | 110 | 192 | 30 | 16 | 17 | 6.8 | 13.2 | 16.0 | 19.8 | 24.5 | 11.3 |
| PLGF-1 | 277 | 74 | 129 | 36 | 25 | — | 13 | 18 | 22 | 24 | 23 | — |
| PLGF-1 + PLGF-2 | 278 | 108 | 187 | 27 | 17 | 17 | 87.8 | 208.9 | 285.0 | 323.4 | 803.4 | 544.8 |
| Protein C Activated | 273 | 65 | 115 | 25 | 15 | 10 | 20.1 | 3.3 | 3.2 | 4.2 | 2.7 | 4.1 |
| Protein C Total | 282 | 116 | 197 | 33 | 16 | 19 | 2.7 | 2.4 | 2.0 | 1.8 | 1.2 | 1.9 |
| Pulmonary surfactant | 274 | 52 | 95 | 22 | 13 | 10 | 0.2 | 0.5 | 0.4 | 0.5 | 0.5 | 0.6 |
| protein A | | | | | | | | | | | | |
| Pulmonary surfactant | 273 | 105 | 182 | 30 | 19 | 14 | 3064.3 | 2308.7 | 2426.1 | 2332.2 | 2269.0 | 4491.1 |
| protein B | | | | | | | | | | | | |
| Pulmonary surfactant | 283 | 112 | 194 | 29 | 17 | 17 | 20.1 | 8.1 | 9.4 | 9.2 | 9.3 | 14.1 |
| protein D | | | | | | | | | | | | |
| PTEN | 278 | 113 | 191 | 31 | 15 | 19 | 0.2 | 0.5 | 1.2 | 1.0 | 1.1 | 1.0 |
| RAGE | 248 | 119 | 199 | 31 | 16 | 20 | 0.5 | 0.4 | 0.9 | 0.5 | 0.8 | 0.7 |
| sICAM1 | 0 | 20 | 30 | 8 | 7 | 5 | — | 638.3 | 743.7 | 951.7 | 901.3 | 642.3 |
| Sphingosine Kinase I | 271 | 103 | 173 | 26 | 18 | 14 | 0.0 | 2.2 | 3.8 | 2.5 | 4.3 | 1.0 |
| TIMP-1 | 277 | 15 | 28 | 10 | 6 | — | 0.2 | 0.3 | 0.3 | 0.3 | 0.4 | — |
| Tissue Factor | 0 | 22 | 33 | 9 | 7 | 6 | — | 4.7 | 2.4 | 27.3 | 3.1 | 0.0 |
| TNF-a | 274 | 22 | 33 | 9 | 7 | 6 | 14.6 | 41.0 | 39.8 | 34.0 | 77.4 | 45.7 |
| sTNFR1a | 274 | 105 | 182 | 31 | 19 | 14 | 532.1 | 1453.1 | 2059.8 | 3849.0 | 5191.1 | 11142.7 |
| sTNFRSF3 | 277 | 31 | 79 | 28 | 20 | — | 1.7 | 3.0 | 3.7 | 7.3 | 6.0 | — |
| (Lymphotoxin | | | | | | | | | | | | |
| B receptor) | | | | | | | | | | | | |
| sTNFRSF7 (CD27) | 277 | 20 | 58 | 15 | 13 | — | 6.7 | 11.1 | 12.3 | 20.3 | 13.9 | — |
| sTNFRSF11A | 217 | 79 | 157 | 34 | 23 | — | <0.28 | 0.4 | <0.28 | 1.1 | 1.3 | — |
| (RANK) | | | | | | | | | | | | |
| sTNFSF14 (LIGHT) | 274 | 40 | 85 | 23 | 12 | 9 | 110.9 | 140.7 | 133.5 | 136.0 | 107.8 | 71.2 |
| sTREM-1 | 274 | 74 | 117 | 17 | 12 | 9 | 0.7 | 1.2 | 1.6 | 2.3 | 4.5 | 9.1 |
| TREM-1sv | 273 | 22 | 33 | 9 | 7 | 5 | 0.0 | 0.2 | 0.1 | 0.2 | 0.3 | 0.2 |
| UCRP | 277 | 20 | 58 | 15 | 13 | — | 0.5 | 2.1 | 1.9 | 3.5 | 3.2 | — |
| uPAR | 273 | 80 | 152 | 29 | 16 | 13 | 5.0 | 10.5 | 11.2 | 17.9 | 25.8 | 19.0 |
| VCAM-1 | 273 | 38 | 80 | 24 | 12 | 10 | 790.2 | 1290.5 | 1489.6 | 1280.2 | 1407.5 | 1694.0 |
| | Concentration | | | | | | Concentration | | | | | |
| | (25^{th} percentile) | | | | | | (75^{th} percentile) | | | | | |
| Adiponectin | 1333 | 3877 | 1421 | 2561 | 1237 | — | 3883 | 7075 | 5785 | 8535 | 8038 | — |
| Adrenomedullin | 39.2 | 157.9 | 198.2 | 398.8 | 268.5 | 544.6 | 143.4 | 487.0 | 659.2 | 1046.1 | 1094.7 | 4495.8 |
| Angiotensinogen | 46.1 | 42.8 | 31.3 | 56.3 | 46.1 | 48.6 | 76.8 | 98.7 | 83.4 | 114.5 | 92.3 | 74.6 |
| Apolipoprotein C1 | 1216 | 783 | 694 | 506 | 709 | — | 2437 | 1376 | 1201 | 1481 | 1369 | — |
| Big Endothelin-1 | <18 | <18 | <18 | 38.3 | 43.3 | 46.6 | <18 | 67.1 | 99.6 | 105.7 | 151.5 | 132.8 |
| BNP₁₋₁₀₈ | — | 6.0 | 5.0 | 19.9 | 122.7 | 296.2 | — | 443.8 | 292.6 | 683.8 | 324.0 | 658.2 |
| BNP₇₉₋₁₀₈ | 0.0 | 0.8 | 0.3 | 0.3 | 6.8 | 2.4 | 9.7 | 32.0 | 25.6 | 16.3 | 17.2 | 19.5 |
| BNP (BNP₇₇₋₁₀₈) | 4.7 | 0.0 | 2.6 | 4.9 | 118.9 | 48.4 | 35.7 | 48.8 | 182.7 | 282.5 | 296.7 | 731.5 |
| BNP₃₋₁₀₈ | 2.6 | 0.0 | 17.7 | 0.0 | 195.7 | 162.4 | 98.8 | 323.1 | 624.4 | 841.1 | 948.5 | 1443.8 |
| Complement C3a | — | 570.8 | 621.2 | 563.3 | 597.2 | 608.5 | — | 997.9 | 1180.0 | 1191.5 | 956.0 | 858.1 |
| Calcitonin | 0.0 | 0.0 | 0.0 | 0.0 | 0.0 | 0.0 | 32.5 | 27.8 | 41.3 | 27.3 | 24.4 | 33.0 |
| Caspase-3 | 0.6 | 3.2 | 3.7 | 4.4 | 3.6 | 4.4 | 1.4 | 14.0 | 21.8 | 31.6 | 17.3 | 11.0 |
| CCL16 | 9.4 | 5.1 | 5.1 | 2.8 | 12.7 | — | 19.8 | 12.3 | 16.1 | 8.1 | 16.2 | — |
| CCL19 | 0.1 | 0.3 | 0.4 | 0.3 | 0.9 | 0.3 | 0.3 | 0.9 | 1.4 | 2.9 | 3.6 | 1.2 |
| CCL20 | 2.3 | 18.2 | 22.2 | 53.6 | 131.5 | 79.1 | 19.2 | 155.4 | 217.1 | 473.0 | 674.7 | 1350.8 |
| CCL23 | 0.1 | 0.2 | 0.5 | 0.3 | 0.5 | 0.8 | 0.2 | 0.8 | 1.5 | 1.7 | 2.4 | 1.6 |
| CCL26 | 13.6 | 11.4 | 13.6 | 13.3 | 19.6 | — | 55.5 | 78.6 | 54.8 | 58.4 | 76.3 | — |
| CCL4 (MIP1β) | 1.2 | 52.8 | 96.9 | 106.0 | 203.6 | 172.9 | 1.2 | 321.1 | 508.2 | 492.8 | 696.8 | 651.7 |
| CCL5 | 0.3 | 10.8 | 14.9 | 2.9 | 3.3 | 4.1 | 2.5 | 85.7 | 137.2 | 62.0 | 25.0 | 13.1 |
| CCL8 | 0.0 | 0.0 | 0.0 | 0.0 | 0.0 | 0.0 | 0.2 | 0.0 | 0.0 | 0.0 | 0.0 | 0.0 |
| CK-BB | 0.2 | 0.0 | 0.0 | 0.0 | 0.0 | 0.0 | 0.8 | 0.2 | 0.3 | 0.4 | 0.3 | 0.2 |
| CK-MB | <1 | <1 | <1 | <1 | <1 | — | 1.5 | 1.3 | 1.2 | 2.1 | 3.7 | — |
| C-reactive protein | 0.0 | 17.6 | 28.2 | 36.9 | 49.2 | 31.3 | 2.6 | 64.8 | 105.4 | 118.6 | 105.2 | 97.2 |
| (CRP) | | | | | | | | | | | | |
| CXCL5 | 44 | 49 | 125 | 58 | 29 | — | 186 | 421 | 579 | 361 | 149 | — |
| CXCL9 | — | 0.1 | 1.0 | 0.3 | 1.8 | — | — | 5.6 | 7.2 | 1.0 | 3.0 | — |
| CXCL13 | 0.0 | 0.0 | 0.0 | 6.7 | 0.0 | 97.8 | 28.5 | 89.6 | 206.3 | 514.0 | 371.9 | 639.5 |
| CXCL16 | 2.3 | 3.6 | 4.4 | 3.0 | 4.0 | 10.1 | 4.2 | 9.1 | 12.2 | 22.3 | 13.3 | 23.0 |
| CXCL6 | 4.8 | 41.7 | 56.7 | 57.3 | 44.7 | 44.7 | 21.6 | 126.4 | 161.0 | 147.0 | 176.8 | 123.3 |
| Cystatin C | <1186 | <1186 | <1186 | 1485 | 1588 | 1595.0 | 1186 | 1414 | 1842 | 4696 | 5170 | 6780.4 |
| D-Dimer | 0.0 | 225.2 | 693.7 | 1697.1 | 969.0 | 1647.0 | 275.2 | 2363.9 | 3345.9 | 5860.5 | 5124.9 | 6323.7 |
| sDR6 | 3.4 | 6.1 | 11.7 | 9.2 | 73.4 | 13.6 | 33.2 | 254.6 | 174.2 | 311.2 | 766.3 | 117.6 |
| Glutathione-S- | 0.0 | 0.0 | 0.2 | 0.5 | 1.1 | 0.0 | 4.5 | 6.5 | 7.9 | 5.8 | 6.9 | 5.7 |
| transferase A (GSTA) | | | | | | | | | | | | |
| HSP-60 | 0.5 | 1.4 | 1.0 | 3.2 | 1.9 | — | 1.5 | 3.4 | 4.8 | 7.0 | 8.4 | — |
| HMG-1 | 0.6 | 1.9 | 1.7 | 1.5 | 1.8 | 3.0 | 4.1 | 5.0 | 6.5 | 5.2 | 7.1 | 7.9 |
| I-FABP | 0.9 | 0.7 | 0.6 | 0.3 | 0.7 | 2.3 | 1.8 | 2.6 | 3.1 | 1.5 | 2.4 | 7.3 |
| IGFBP-1 | 17.4 | 12.0 | 10.0 | 15.3 | 30.4 | — | 100.9 | 277.6 | 100.5 | 367.2 | 227.2 | — |
| IL-10 | 0.0 | 1.2 | 4.6 | 21.3 | 41.5 | 13.5 | 25.4 | 64.9 | 102.7 | 105.4 | 191.5 | 144.7 |
| IL-1β | 0.0 | 0.1 | 0.1 | 0.1 | 0.1 | 0.1 | 84.8 | 85.4 | 92.5 | 61.9 | 85.6 | 0.1 |
| IL-1ra | 156.5 | 194.0 | 280.6 | 499.9 | 711.0 | 371.9 | 327.7 | 1003.6 | 1480.3 | 3890.9 | 9351.8 | 5759.2 |
| IL-22 | 0.0 | 0.0 | 0.0 | 2.3 | 0.0 | 0.0 | 21.3 | 42.5 | 48.5 | 66.6 | 115.8 | 72.8 |
| IL2sRA | 0.4 | 0.7 | 0.8 | 1.0 | 1.7 | 1.0 | 0.6 | 1.7 | 2.4 | 3.2 | 5.0 | 3.1 |
| IL-6 | 0.0 | 0.0 | 23.4 | 41.2 | 22.2 | 26.0 | 7.1 | 295.3 | 955.3 | 1435.7 | 1406.8 | 1514.2 |
| IL-8 | 0.0 | 0.0 | 0.0 | 0.0 | 0.0 | 0.0 | 8.5 | 0.0 | 3.5 | 0.0 | 17.8 | 0.0 |
| MCP-1 | 23.8 | 31.3 | 40.6 | 51.0 | 69.1 | 81.8 | 35.4 | 84.2 | 135.5 | 140.8 | 266.2 | 304.0 |
| MIF | 10.4 | 30.3 | 34.5 | 35.6 | 61.8 | 71.3 | 19.1 | 110.1 | 108.1 | 93 | 116.3 | 103.9 |
| MMP9 | 14.8 | 29.9 | 20.6 | 7.9 | 7.6 | 11.5 | 28.4 | 312.9 | 266.3 | 226.5 | 147.6 | 160.8 |
| MPO | 7.9 | 15.9 | 32.0 | 41.1 | 61.1 | 76.6 | 31.1 | 83.9 | 136.8 | 130.7 | 262.3 | 163.2 |
| MYOGLOBIN | 51.7 | 55.1 | 58.1 | 92.0 | 175.6 | 217.4 | 94.2 | 206.9 | 310.8 | 802.9 | 1097.7 | 1031.0 |
| NGAL | 214 | 554 | 923 | 1000 | 551 | — | 704 | 1000 | 1000 | 1000 | 1000 | 1000 |
| PAI-1 | 2.1 | 7.8 | 8.5 | 8.4 | 12.8 | 8.7 | 14.7 | 29.0 | 26.5 | 74.9 | 83.8 | 68.9 |
| PLGF-1 | <10 | <10 | <10 | <10 | <10 | — | 45 | 77 | 74 | 53 | 87 | — |
| PLGF-1 + _PLGF-2 | 57.1 | 115.7 | 145.2 | 199.9 | 528.6 | 325.5 | 137.7 | 333.1 | 559.5 | 817.3 | 2113.4 | 2038.5 |
| Protein C Activated | 15.5 | 0.1 | 0.2 | 1.1 | 0.4 | 1.8 | 25.1 | 5.8 | 5.5 | 7.0 | 4.0 | 6.0 |
| Protein C Total | 2.3 | 1.8 | 1.2 | 0.8 | 0.9 | 0.9 | 3.3 | 3.1 | 2.8 | 2.6 | 1.4 | 2.7 |
| Pulmonary surfactant | 0.1 | 0.2 | 0.2 | 0.2 | 0.3 | 0.3 | 0.4 | 0.9 | 0.8 | 0.8 | 0.7 | 0.8 |
| protein A | | | | | | | | | | | | |
| Pulmonary surfactant | 1884.9 | 1127.2 | 1081.4 | 1108.7 | 961.2 | 2834.2 | 4433.8 | 4921.3 | 5277.1 | 6687.7 | 5399.0 | 5444.9 |
| protein B | | | | | | | | | | | | |
| Pulmonary surfactant | 12.0 | 3.9 | 4.1 | 4.2 | 4.3 | 3.1 | 30.2 | 13.9 | 20.1 | 18.1 | 12.0 | 22.1 |
| protein D | | | | | | | | | | | | |
| PTEN | 0.0 | 0.0 | 0.1 | 0.1 | 0.1 | 0.0 | 0.6 | 1.3 | 3.6 | 4.4 | 3.1 | 2.1 |
| RAGE | 0.0 | 0.0 | 0.0 | 0.1 | 0.0 | 0.2 | 1.4 | 1.5 | 1.9 | 1.7 | 1.1 | 2.9 |
| sICAM1 | — | 524.8 | 519.1 | 601.2 | 611.2 | 638.0 | — | 938.3 | 1358.1 | 1425.8 | 1361.5 | 705.7 |
| Sphingosine Kinase I | 0.0 | 0.5 | 0.4 | 0.2 | 1.0 | 0.4 | 1.5 | 9.0 | 12.6 | 7.8 | 13.6 | 1.9 |
| TIMP-1 | 0.2 | 0.2 | 0.2 | 0.3 | 0.3 | — | 0.3 | 0.3 | 0.3 | 0.3 | 0.4 | — |
| Tissue Factor | — | 0.0 | 0.0 | 3.9 | 0.8 | 0.0 | — | 40.5 | 35.4 | 35.8 | 30.3 | 0.0 |
| TNF-a | 9.6 | 18.0 | 25.7 | 21.1 | 43.3 | 36.0 | 28.2 | 102.6 | 84.3 | 56.4 | 88.6 | 80.6 |
| sTNFR1a | 398.5 | 1150.7 | 1280.8 | 1938.7 | 3089.1 | 3643.0 | 661.4 | 2620.6 | 4661.5 | 10153.8 | 13477.8 | 24560.5 |
| sTNFRSF3 | 1.4 | 2.3 | 2.5 | 4.1 | 4.0 | — | 2.1 | 4.1 | 6.1 | 13.0 | 7.7 | — |
| (Lymphotoxin | | | | | | | | | | | | |
| B receptor) | | | | | | | | | | | | |
| sTNFRSF7 (CD27) | 5.3 | 9.4 | 8.9 | 11.8 | 12.0 | — | 8.2 | 18.7 | 15.8 | 50.3 | 35.0 | — |
| sTNFRSF11A | <0.28 | <0.28 | <0.28 | <0.28 | <0.28 | — | <0.28 | 0.7 | 0.6 | 2.9 | 2.6 | — |
| (RANK) | | | | | | | | | | | | |
| sTNFSF14 (LIGHT) | 61.9 | 82.7 | 62.0 | 86.6 | 20.8 | 41.4 | 222.3 | 339.3 | 239.7 | 274.1 | 295.3 | 274.7 |
| sTREM-1 | 0.5 | 0.6 | 0.8 | 1.7 | 1.7 | 4.9 | 1.0 | 2.3 | 3.5 | 4.9 | 8.9 | 16.2 |
| TREM-1sv | 0.0 | 0.1 | 0.0 | 0.1 | 0.2 | 0.1 | 0.2 | 0.7 | 0.4 | 0.4 | 0.4 | 0.2 |
| uPAR | 4.3 | 7.7 | 8.0 | 10.9 | 17.0 | 9.4 | 6.3 | 14.6 | 16.9 | 31.6 | 36.8 | 67.1 |
| UCRP | 0.4 | 1.2 | 0.9 | 1.4 | 1.0 | — | 0.9 | 4.8 | 8.3 | 8.9 | 19.6 | — |
| VCAM-1 | 507.2 | 1019.6 | 1198.0 | 1031.8 | 1101.5 | 1298.6 | 1038.0 | 1785.3 | 1812.6 | 1467.7 | 1672.4 | 1795.8 |

Using this data, ROC analysis was performed to compare various groups, labeled for convenience as “control” and “disease.” In the prognosis groups described below, subjects considered were all patients diagnosed as having systemic inflammatory response syndrome (SIRS), sepsis, severe sepsis, septic shock or multiple organ dysfunction syndrome (MODS), which were divided into groups based on 30-day mortality. As discussed herein, preferred markers for distinguishing two diagnosis groups provide a ROC curve area of at least 0.6, more preferably 0.7, still more preferably at least 0.8, even more preferably at least 0.9, and most preferably at least 0.95. These preferred markers may be used individually or as part of a marker panel as described herein.

TABLE 2

| | Univariate ROC area | | | | | Univariate ROC area | | | | |
|---|---|---|---|---|---|---|---|---|---|---|
| | | “disease” | | | | | “disease” | | | |
| | | N (sepsis/ | | | | | N (sepsis/ | | | |
| | | severe | | | | | severe | | | |
| | | sepsis/ | | | | | sepsis/ | | | |
| | | septic | | | | | septic | | | |
| | “control” N | shock/ | ROC | | Change with | “control” | shock/ | ROC | | Change with |
| | (normal) | MODS) | area | p-value | disease | N (SIRS) | MODS) | area | p-value | disease |
| Adiponectin | 277 | 86 | 0.581 | 1.77E−02 | Increase | 20 | 86 | 0.663 | 2.50E−03 | Decrease |
| Adrenomedullin | 274 | 229 | 0.880 | <1.0E−03 | Increase | 90 | 229 | 0.627 | <1.0E−03 | Increase |
| Angiotensinogen | 273 | 129 | 0.507 | 4.22E−01 | Decrease | 40 | 129 | 0.543 | 2.08E−01 | Decrease |
| Apolipoprotein C1 | 277 | 64 | 0.772 | <1.0E−03 | Decrease | 14 | 64 | 0.522 | 2.50E−01 | Decrease |
| Big Endothelin-1 | 277 | 190 | 0.816 | <1.0E−03 | Increase | 74 | 190 | 0.632 | <1.0E−03 | Increase |
| BNP₁₋₁₀₈ | | | | | | 20 | 53 | 0.539 | 3.07E−01 | Increase |
| BNP₇₉₋₁₀₈ | 273 | 53 | 0.688 | <1.0E−03 | Increase | 20 | 53 | 0.535 | 3.27E−01 | Decrease |
| BNP (BNP₇₇₋₁₀₈) | 252 | 265 | 0.666 | <1.0E−03 | Increase | 120 | 265 | 0.671 | <1.0E−03 | Increase |
| BNP₃₋₁₀₈ | 278 | 250 | 0.707 | <1.0E−03 | Increase | 116 | 250 | 0.620 | <1.0E−03 | Increase |
| Complement C3a | | | | | | 53 | 146 | 0.541 | 1.85E−01 | Increase |
| Calcitonin | 277 | 257 | 0.525 | 1.59E−01 | Decrease | 114 | 257 | 0.521 | 2.55E−01 | Increase |
| Caspase-3 | 279 | 260 | 0.931 | <1.0E−03 | Increase | 112 | 260 | 0.573 | 1.07E−02 | Increase |
| CCL16 | 277 | 25 | 0.647 | 1.36E−02 | Decrease | 7 | 25 | 0.571 | 2.92E−01 | Increase |
| CCL19 | 275 | 259 | 0.847 | <1.0E−03 | Increase | 115 | 259 | 0.591 | 1.50E−03 | Increase |
| CCL20 | 274 | 246 | 0.888 | <1.0E−03 | Increase | 105 | 246 | 0.620 | <1.0E−03 | Increase |
| CCL23 | 279 | 257 | 0.928 | <1.0E−03 | Increase | 110 | 257 | 0.683 | <1.0E−03 | Increase |
| CCL26 | 82 | 64 | 0.510 | 4.14E−01 | Increase | 14 | 64 | 0.509 | 4.63E−01 | Increase |
| CCL4 (MIP1β) | 273 | 244 | 0.852 | <1.0E−03 | Increase | 103 | 244 | 0.592 | 2.29E−03 | Increase |
| CCL5 | 277 | 179 | 0.889 | <1.0E−03 | Increase | 89 | 179 | 0.516 | 3.29E−01 | Increase |
| CCL8 | 276 | 254 | 0.631 | <1.0E−03 | Decrease | 109 | 254 | 0.501 | 4.86E−01 | Decrease |
| CK-BB | 258 | 263 | 0.780 | <1.0E−03 | Decrease | 118 | 263 | 0.537 | 1.17E−01 | Increase |
| CK-MB | 215 | 216 | 0.506 | 4.20E−01 | Decrease | 77 | 216 | 0.517 | 3.32E−01 | Increase |
| C-reactive protein (CRP) | 265 | 255 | 0.980 | <1.0E−03 | Increase | 117 | 255 | 0.631 | <1.0E−03 | Increase |
| CXCL5 | 277 | 646 | 0.64 | <1.0E−03 | Increase | 14 | 64 | 0.550 | 2.76E−01 | Increase |
| CXCL9 | 11 | 37 | 0.523 | 4.19E−01 | Increase | | | | | |
| CXCL13 | 278 | 255 | 0.712 | <1.0E−03 | Increase | 110 | 255 | 0.642 | <1.0E−03 | Increase |
| CXCL16 | 284 | 184 | 0.827 | <1.0E−03 | Increase | 91 | 184 | 0.608 | 9.73E−04 | Increase |
| CXCL6 | 273 | 244 | 0.916 | <1.0E−03 | Increase | 103 | 244 | 0.567 | 2.10E−02 | Increase |
| Cystatin C | 220 | 217 | 0.633 | <1.0E−03 | Increase | 83 | 217 | 0.614 | <1.0E−03 | Increase |
| D-Dimer | 248 | 268 | 0.922 | <1.0E−03 | Increase | 119 | 268 | 0.636 | <1.0E−03 | Increase |
| sDR6 | 272 | 243 | 0.686 | <1.0E−03 | Increase | 105 | 243 | 0.549 | 8.12E−02 | Increase |
| Glutathione-S-transferase | 271 | 231 | 0.598 | <1.0E−03 | Increase | 103 | 231 | 0.527 | 2.12E−01 | Increase |
| A (GSTA) | | | | | | | | | | |
| HSP-60 | 277 | 86 | 0.795 | <1.0E−03 | Increase | 20 | 86 | 0.558 | 1.99E−01 | Increase |
| HMG-1 | 277 | 262 | 0.697 | <1.0E−03 | Increase | 111 | 262 | 0.540 | 1.01E−01 | Increase |
| I-FABP | 273 | 55 | 0.500 | 4.96E−01 | Decrease | 22 | 55 | 0.514 | 4.21E−01 | Decrease |
| IGFBP-1 | 277 | 122 | 0.503 | 4.61E−01 | Decrease | 31 | 122 | 0.581 | 9.09E−02 | Decrease |
| IL-10 | 274 | 240 | 0.719 | <1.0E−03 | Increase | 100 | 240 | 0.595 | 2.35E−03 | Increase |
| IL-1β | 274 | 93 | 0.550 | 5.28E−02 | Increase | 35 | 93 | 0.552 | 1.89E−01 | Decrease |
| IL-1ra | 256 | 268 | 0.812 | <1.0E−03 | Increase | 120 | 268 | 0.619 | <1.0E−03 | Increase |
| IL-22 | 280 | 257 | 0.590 | <1.0E−03 | Increase | 115 | 257 | 0.549 | 6.16E−02 | Increase |
| IL2sRA | 274 | 210 | 0.877 | <1.0E−03 | Increase | 80 | 210 | 0.635 | <1.0E−03 | Increase |
| IL-6 | 281 | 258 | 0.846 | <1.0E−03 | Increase | 113 | 258 | 0.627 | <1.0E−03 | Increase |
| IL-8 | 263 | 268 | 0.600 | <1.0E−03 | Decrease | 119 | 268 | 0.539 | 1.06E−01 | Increase |
| MCP-1 | 274 | 146 | 0.876 | <1.0E−03 | Increase | 53 | 146 | 0.609 | 6.34E−03 | Increase |
| MIF | 277 | 144 | 0.927 | <1.0E−03 | Increase | 56 | 144 | 0.562 | 9.94E−02 | Increase |
| MMP9 | 270 | 257 | 0.708 | <1.0E−03 | Increase | 114 | 257 | 0.548 | 6.29E−02 | Decrease |
| MPO | 258 | 262 | 0.793 | <1.0E−03 | Increase | 116 | 262 | 0.641 | <1.0E−03 | Increase |
| Myoglobin | 264 | 266 | 0.726 | <1.0E−03 | Increase | 118 | 266 | 0.608 | <1.0E−03 | Increase |
| NGAL | 221 | 219 | 0.808 | <1.0E−03 | Increase | 195 | 24 | 0.528 | 3.32E−01 | Decrease |
| PAI-1 | 278 | 255 | 0.712 | <1.0E−03 | Increase | 110 | 255 | 0.546 | 7.74E−02 | Increase |
| PLGF-1 | 277 | 190 | 0.578 | 1.94E−03 | Increase | 192 | 26 | 0.577 | 8.97E−02 | Decrease |
| PLGF-1 + PLGF-2 | 278 | 248 | 0.847 | <1.0E−03 | Increase | 108 | 248 | 0.628 | <1.0E−03 | Increase |
| Protein C Activated | 273 | 165 | 0.980 | <1.0E−03 | Decrease | 65 | 165 | 0.506 | 4.40E−01 | Increase |
| Protein C Total | 282 | 265 | 0.719 | <1.0E−03 | Decrease | 116 | 265 | 0.617 | <1.0E−03 | Decrease |
| Pulmonary surfactant | 274 | 140 | 0.702 | <1.0E−03 | Increase | 52 | 140 | 0.524 | 3.08E−01 | Decrease |
| protein A | | | | | | | | | | |
| Pulmonary surfactant | 273 | 245 | 0.551 | 2.77E−02 | Decrease | 105 | 245 | 0.512 | 3.61E−01 | Increase |
| protein B | | | | | | | | | | |
| Pulmonary surfactant | 283 | 257 | 0.694 | <1.0E−03 | Decrease | 112 | 257 | 0.541 | 9.76E−02 | Increase |
| protein D | | | | | | | | | | |
| PTEN | 278 | 256 | 0.705 | <1.0E−03 | Increase | 113 | 256 | 0.621 | <1.0E−03 | Increase |
| RAGE | 248 | 266 | 0.544 | 4.22E−02 | Increase | 119 | 266 | 0.576 | 8.95E−03 | Increase |
| sICAM1 | | | | | | 20 | 50 | 0.589 | 9.57E−02 | Increase |
| Sphingosine Kinase I | 271 | 231 | 0.752 | <1.0E−03 | Increase | 103 | 231 | 0.546 | 8.32E−02 | Increase |
| TIMP-1 | 277 | 44 | 0.715 | <1.0E−03 | Increase | 15 | 44 | 0.529 | 3.69E−01 | Increase |
| Tissue Factor | | | | | | 22 | 55 | 0.534 | 3.25E−01 | Decrease |
| TNF-a | 274 | 55 | 0.774 | <1.0E−03 | Increase | 22 | 55 | 0.508 | 4.59E−01 | Increase |
| sTNFR1a | 274 | 246 | 0.953 | <1.0E−03 | Increase | 105 | 246 | 0.658 | <1.0E−03 | Increase |
| sTNFRSF3 (Lymphotoxin | 277 | 127 | 0.908 | <1.0E−03 | Increase | 31 | 127 | 0.683 | <1.0E−03 | Increase |
| B Receptor) | | | | | | | | | | |
| sTNFRSF7 (CD27) | 277 | 86 | 0.831 | <1.0E−03 | Increase | 20 | 86 | 0.529 | 3.35E−01 | Increase |
| sTNFRSF11A (RANK) | 217 | 214 | 0.705 | <1.0E−03 | Increase | 79 | 214 | 0.547 | 9.18E−02 | Increase |
| sTNFSF14 (LIGHT) | 274 | 129 | 0.505 | 4.41E−01 | Increase | 40 | 129 | 0.552 | 1.58E−01 | Decrease |
| sTREM-1 | 274 | 155 | 0.825 | <1.0E−03 | Increase | 74 | 155 | 0.635 | <1.0E−03 | Increase |
| TREM-1sv | 273 | 54 | 0.694 | <1.0E−03 | Increase | 22 | 54 | 0.568 | 1.88E−01 | Decrease |
| UCRP | 277 | 86 | 0.866 | <1.0E−03 | Increase | 20 | 86 | 0.508 | 4.51E−01 | Increase |
| uPAR | 273 | 210 | 0.909 | <1.0E−03 | Increase | 80 | 210 | 0.591 | 5.49E−03 | Increase |
| VCAM-1 | 273 | 126 | 0.878 | <1.0E−03 | Increase | 38 | 126 | 0.561 | 1.43E−01 | Increase |

| | Univariate ROC area | | | | | Univariate ROC area | | | | |
|---|---|---|---|---|---|---|---|---|---|---|
| | | “disease” | | | | | “disease” | | | |
| | | N (culture | | | | | N (culture | | | |
| | | positive | | | | | negative | | | |
| | | sepsis/ | | | | | sepsis/ | | | |
| | | severe | | | | | severe | | | |
| | | sepsis/ | | | | | sepsis/ | | | |
| | | septic | | | | | septic | | | |
| | “control” | shock/ | ROC | | Change with | “control” | shock/ | ROC | | Change with |
| | N (SIRS) | MODS) | area | p-value | disease | N (SIRS) | MODS) | area | p-value | disease |
| Adrenomedullin | 90 | 37 | 0.651 | 4.36E−03 | Increase | 90 | 192 | 0.622 | <1.0E−03 | Increase |
| Angiotensinogen | 40 | 21 | 0.555 | 2.32E−01 | Increase | 40 | 108 | 0.562 | 1.23E−01 | Decrease |
| Big Endothelin-1 | 78 | 24 | 0.607 | 5.74E−02 | Increase | 78 | 158 | 0.572 | 3.91E−02 | Increase |
| BNP₁₋₁₀₈ | 20 | 9 | 0.517 | 4.47E−01 | Increase | 20 | 44 | 0.544 | 2.93E−01 | Increase |
| BNP₇₉₋₁₀₈ | 20 | 9 | 0.594 | 2.25E−01 | Decrease | 20 | 44 | 0.523 | 3.89E−01 | Decrease |
| BNP (BNP₇₇₋₁₀₈) | 120 | 33 | 0.797 | <1.0E−03 | Increase | 120 | 232 | 0.653 | <1.0E−03 | Increase |
| BNP₃₋₁₀₈ | 116 | 33 | 0.649 | 4.94E−03 | Increase | 116 | 217 | 0.615 | <1.0E−03 | Increase |
| Complement C3a | 53 | 21 | 0.506 | 4.68E−01 | Decrease | 53 | 125 | 0.549 | 1.47E−01 | Increase |
| Calcitonin | 114 | 35 | 0.529 | 3.09E−01 | Increase | 114 | 222 | 0.520 | 2.74E−01 | Increase |
| Caspase-3 | 112 | 33 | 0.676 | <1.0E−03 | Increase | 112 | 227 | 0.558 | 3.75E−02 | Increase |
| CCL19 | 115 | 35 | 0.619 | 1.05E−02 | Increase | 115 | 224 | 0.586 | 2.96E−03 | Increase |
| CCL20 | 105 | 37 | 0.681 | <1.0E−03 | Increase | 105 | 209 | 0.610 | <1.0E−03 | Increase |
| CCL23 | 110 | 33 | 0.723 | <1.0E−03 | Increase | 110 | 224 | 0.677 | <1.0E−03 | Increase |
| CCL4 (MIP1β) | 103 | 37 | 0.656 | 1.43E−03 | Increase | 103 | 207 | 0.581 | 8.11E−03 | Increase |
| CCL5 | 89 | 22 | 0.503 | 4.86E−01 | Increase | 89 | 157 | 0.518 | 3.15E−01 | Increase |
| CCL8 | 109 | 32 | 0.511 | 4.27E−01 | Decrease | 109 | 222 | 0.500 | 4.98E−01 | Increase |
| CK-BB | 118 | 35 | 0.524 | 3.43E−01 | Increase | 118 | 228 | 0.539 | 1.11E−01 | Increase |
| C-reactive protein (CRP) | 117 | 33 | 0.715 | <1.0E−03 | Increase | 117 | 222 | 0.618 | <1.0E−03 | Increase |
| CXCL13 | 110 | 32 | 0.721 | <1.0E−03 | Increase | 110 | 223 | 0.631 | <1.0E−03 | Increase |
| CXCL16 | 91 | 22 | 0.656 | 1.71E−02 | Increase | 91 | 162 | 0.601 | 2.47E−03 | Increase |
| CXCL6 | 103 | 36 | 0.584 | 6.22E−02 | Increase | 103 | 208 | 0.565 | 2.87E−02 | Increase |
| Cystatin C | 38 | 18 | 0.531 | 3.72E−01 | Increase | 38 | 108 | 0.583 | 6.06E−02 | Increase |
| D-Dimer | 119 | 34 | 0.673 | <1.0E−03 | Increase | 119 | 234 | 0.630 | <1.0E−03 | Increase |
| sDR6 | 105 | 37 | 0.547 | 1.84E−01 | Increase | 105 | 206 | 0.549 | 8.37E−02 | Increase |
| Glutathione-S-transferase | 103 | 37 | 0.520 | 3.54E−01 | Increase | 103 | 194 | 0.529 | 2.07E−01 | Increase |
| A (GSTA) | | | | | | | | | | |
| HMG-1 | 111 | 35 | 0.530 | 2.95E−01 | Increase | 111 | 227 | 0.542 | 9.79E−02 | Increase |
| I-FABP | 22 | 9 | 0.573 | 2.77E−01 | Increase | 22 | 46 | 0.531 | 3.37E−01 | Decrease |
| IL-10 | 100 | 36 | 0.609 | 3.12E−02 | Increase | 100 | 204 | 0.593 | 3.84E−03 | Increase |
| IL-1β | 35 | 18 | 0.567 | 2.17E−01 | Decrease | 35 | 75 | 0.548 | 2.14E−01 | Decrease |
| IL-1ra | 120 | 35 | 0.655 | 1.33E−03 | Increase | 120 | 233 | 0.614 | <1.0E−03 | Increase |
| IL-22 | 115 | 34 | 0.586 | 6.54E−02 | Increase | 115 | 223 | 0.544 | 9.22E−02 | Increase |
| IL2sRA | 80 | 34 | 0.749 | <1.0E−03 | Increase | 80 | 176 | 0.613 | 1.03E−03 | Increase |
| IL-6 | 113 | 33 | 0.590 | 5.50E−02 | Increase | 113 | 225 | 0.632 | <1.0E−03 | Increase |
| IL-8 | 119 | 34 | 0.603 | 3.62E−02 | Increase | 119 | 234 | 0.529 | 1.78E−01 | Increase |
| MCP-1 | 53 | 21 | 0.604 | 8.96E−02 | Increase | 53 | 125 | 0.610 | 7.37E−03 | Increase |
| MIF | 49 | 22 | 0.645 | 1.53E−02 | Increase | 49 | 97 | 0.533 | 2.68E−01 | Increase |
| MMP9 | 114 | 35 | 0.606 | 3.89E−02 | Decrease | 114 | 222 | 0.539 | 1.16E−01 | Decrease |
| MPO | 116 | 34 | 0.677 | <1.0E−03 | Increase | 116 | 228 | 0.635 | <1.0E−03 | Increase |
| Myoglobin | 118 | 35 | 0.663 | 1.24E−03 | Increase | 118 | 231 | 0.600 | <1.0E−03 | Increase |
| PAI-1 | 110 | 33 | 0.574 | 1.03E−01 | Increase | 110 | 222 | 0.542 | 1.04E−01 | Increase |
| PLGF | 108 | 33 | 0.736 | <1.0E−03 | Increase | 108 | 215 | 0.612 | <1.0E−03 | Increase |
| Protein C Activated | 65 | 24 | 0.555 | 2.13E−01 | Increase | 65 | 141 | 0.502 | 4.83E−01 | Decrease |
| Protein C Total | 116 | 35 | 0.623 | 1.09E−02 | Decrease | 116 | 230 | 0.616 | <1.0E−03 | Decrease |
| Pulmonary surfactant | 52 | 20 | 0.525 | 3.69E−01 | Increase | 52 | 120 | 0.532 | 2.54E−01 | Decrease |
| protein A | | | | | | | | | | |
| Pulmonary surfactant | 105 | 37 | 0.564 | 1.11E−01 | Increase | 105 | 208 | 0.503 | 4.71E−01 | Increase |
| protein B | | | | | | | | | | |
| Pulmonary surfactant | 112 | 33 | 0.538 | 2.65E−01 | Increase | 112 | 224 | 0.542 | 1.02E−01 | Increase |
| protein D | | | | | | | | | | |
| PTEN | 113 | 35 | 0.678 | <1.0E−03 | Increase | 113 | 221 | 0.612 | <1.0E−03 | Increase |
| RAGE | 119 | 35 | 0.601 | 2.89E−02 | Increase | 119 | 231 | 0.572 | 1.38E−02 | Increase |
| sICAM1 | 20 | 8 | 0.569 | 3.08E−01 | Decrease | 20 | 42 | 0.619 | 4.66E−02 | Increase |
| Sphingosine Kinase I | 103 | 37 | 0.605 | 2.82E−02 | Increase | 103 | 194 | 0.535 | 1.56E−01 | Increase |
| Tissue Factor | 22 | 9 | 0.616 | 1.54E−01 | Decrease | 22 | 46 | 0.518 | 4.08E−01 | Decrease |
| TNF-a | 22 | 9 | 0.510 | 4.63E−01 | Decrease | 22 | 46 | 0.512 | 4.42E−01 | Increase |
| TNFR1a | 105 | 37 | 0.750 | <1.0E−03 | Increase | 105 | 209 | 0.642 | <1.0E−03 | Increase |
| sTNFR14 (LIGHT) | 40 | 21 | 0.552 | 2.65E−01 | Decrease | 40 | 108 | 0.552 | 1.66E−01 | Decrease |
| sTREM-1 | 74 | 25 | 0.734 | <1.0E−03 | Increase | 74 | 130 | 0.615 | 2.44E−03 | Increase |
| TREM-1sv | 22 | 9 | 0.588 | 2.13E−01 | Decrease | 22 | 45 | 0.564 | 2.08E−01 | Decrease |
| uPAR | 80 | 34 | 0.660 | 2.78E−03 | Increase | 80 | 176 | 0.578 | 1.84E−02 | Increase |
| VCAM-1 | 38 | 18 | 0.599 | 1.02E−01 | Increase | 38 | 108 | 0.555 | 1.73E−01 | Increase |
| | | | | | | Univariate ROC area | | | | |
| | | | | | | | “disease” | | | |
| | | | | | | | N (severe | | | |
| | | | | | | | sepsis/ | | | |
| | Univariate ROC area | | | | | | septic | | | |
| | “control” | “disease” | ROC | | Change with | “control” N | shock/ | ROC | | Change with |
| | N (SIRS) | N (sepsis) | area | p-value | disease | (sepsis) | MODS) | area | p-value | disease |
| Adrenomedullin | 90 | 168 | 0.577 | 1.71E−02 | Increase | 168 | 61 | 0.690 | <1.0E−03 | Increase |
| Angiotensinogen | 40 | 85 | 0.593 | 4.67E−02 | Decrease | 85 | 44 | 0.653 | 1.05E−03 | Increase |
| Big Endothelin-1 | 78 | 128 | 0.547 | 1.29E−01 | Increase | 128 | 54 | 0.603 | 9.46E−03 | Increase |
| BNP₁₋₁₀₈ | 20 | 32 | 0.505 | 4.74E−01 | Increase | 32 | 21 | 0.599 | 1.13E−01 | Increase |
| BNP₇₉₋₁₀₈ | 20 | 32 | 0.541 | 3.10E−01 | Decrease | 32 | 21 | 0.529 | 3.60E−01 | Increase |
| BNIP (BNP₇₇₋₁₀₈₎ | 120 | 197 | 0.645 | <1.0E−03 | Increase | 197 | 68 | 0.625 | <1.0E−03 | Increase |
| BNP₃₋₁₀₈ | 116 | 184 | 0.605 | 8.46E−04 | Increase | 184 | 66 | 0.573 | 4.35E−02 | Increase |
| Complement C3a | 53 | 98 | 0.573 | 6.55E−02 | Increase | 98 | 48 | 0.593 | 2.97E−02 | Decrease |
| Calcitonin | 114 | 191 | 0.525 | 2.28E−01 | Increase | 191 | 66 | 0.511 | 3.98E−01 | Decrease |
| Caspase-3 | 112 | 196 | 0.570 | 1.76E−02 | Increase | 196 | 64 | 0.511 | 3.96E−01 | Increase |
| CCL19 | 115 | 193 | 0.580 | 7.82E−03 | Increase | 193 | 66 | 0.551 | 1.24E−01 | Increase |
| CCL20 | 105 | 182 | 0.572 | 2.07E−02 | Increase | 182 | 64 | 0.694 | <1.0E−03 | Increase |
| CCL23 | 110 | 194 | 0.681 | <1.0E−03 | Increase | 194 | 63 | 0.538 | 2.02E−01 | Increase |
| CCL4 (MIP1β) | 103 | 181 | 0.576 | 1.46E−02 | Increase | 181 | 63 | 0.561 | 7.43E−02 | Increase |
| CCL5 | 89 | 133 | 0.572 | 3.13E−02 | Increase | 133 | 46 | 0.709 | <1.0E−03 | Decrease |
| CCL8 | 109 | 193 | 0.505 | 4.46E−01 | Increase | 193 | 61 | 0.524 | 2.84E−01 | Decrease |
| CK-BB | 118 | 195 | 0.540 | 1.13E−01 | Increase | 195 | 68 | 0.506 | 4.40E−01 | Decrease |
| C-reactive protein (CRP) | 117 | 191 | 0.621 | <1.0E−03 | Increase | 191 | 64 | 0.538 | 1.80E−01 | Increase |
| CXCL13 | 110 | 192 | 0.614 | <1.0E−03 | Increase | 192 | 63 | 0.638 | <1.0E−03 | Increase |
| CXCL16 | 91 | 137 | 0.589 | 9.89E−03 | Increase | 137 | 47 | 0.601 | 2.91E−02 | Increase |
| CXCL6 | 103 | 181 | 0.574 | 1.79E−02 | Increase | 181 | 63 | 0.520 | 3.24E−01 | Decrease |
| Cystatin C | 38 | 80 | 0.528 | 3.12E−01 | Increase | 80 | 46 | 0.636 | 4.89E−03 | Increase |
| D-Dimer | 119 | 200 | 0.604 | <1.0E−03 | Increase | 200 | 68 | 0.651 | <1.0E−03 | Increase |
| sDR6 | 105 | 182 | 0.535 | 1.73E−01 | Increase | 182 | 61 | 0.577 | 3.90E−02 | Increase |
| Glutathione-S-transferase | 103 | 171 | 0.525 | 2.47E−01 | Increase | 171 | 60 | 0.513 | 3.81E−01 | Increase |
| A (GSTA) | | | | | | | | | | |
| HMG-1 | 111 | 194 | 0.540 | 1.19E−01 | Increase | 194 | 68 | 0.505 | 4.54E−01 | Increase |
| I-FABP | 22 | 33 | 0.538 | 3.18E−01 | Decrease | 33 | 22 | 0.567 | 2.02E−01 | Increase |
| IL-10 | 100 | 179 | 0.566 | 3.10E−02 | Increase | 179 | 61 | | 3.16E−03 | Increase |
| IL-1β | 35 | 57 | 0.515 | 4.07E−01 | Decrease | 57 | 36 | 0.599 | 5.12E−02 | Decrease |
| IL-1ra | 120 | 200 | 0.585 | 5.36E−03 | Increase | 200 | 68 | 0.654 | <1.0E−03 | Increase |
| IL-22 | 115 | 190 | 0.530 | 1.88E−01 | Increase | 190 | 67 | 0.572 | 3.84E−02 | Increase |
| IL2sRA | 80 | 152 | 0.603 | 3.56E−03 | Increase | 152 | 58 | 0.627 | 2.36E−03 | Increase |
| IL-6 | 113 | 192 | 0.620 | <1.0E−03 | Increase | 192 | 66 | 0.534 | 2.11E−01 | Increase |
| IL-8 | 119 | 200 | 0.547 | 7.80E−02 | Increase | 200 | 68 | 0.527 | 2.54E−01 | Decrease |
| MCP-1 | 53 | 98 | 0.568 | 7.87E−02 | Increase | 98 | 48 | 0.631 | 3.11E−03 | Increase |
| MIF | 49 | 87 | 0.552 | 1.69E−01 | Increase | 87 | 32 | 0.505 | 4.66E−01 | Decrease |
| MMP9 | 114 | 190 | 0.530 | 1.87E−01 | Decrease | 190 | 67 | 0.568 | 5.40E−02 | Decrease |
| MPO | 116 | 196 | 0.622 | <1.0E−03 | Increase | 196 | 66 | 0.585 | 1.69E−02 | Increase |
| Myoglobin | 118 | 198 | 0.566 | 2.25E−02 | Increase | 198 | 68 | 0.673 | <1.0E−03 | Increase |
| PAI-1 | 110 | 192 | 0.530 | 1.98E−01 | Increase | 192 | 63 | 0.576 | 4.90E−02 | Increase |
| PLGF | 108 | 187 | 0.592 | 3.51E−03 | Increase | 187 | 61 | 0.652 | <1.0E−03 | Increase |
| Protein C Activated | 65 | 115 | 0.507 | 4.38E−01 | Decrease | 115 | 50 | 0.543 | 1.93E−01 | Increase |
| Protein C Total | 116 | 197 | 0.594 | 2.10E−03 | Decrease | 197 | 68 | 0.597 | 8.58E−03 | Decrease |
| Pulmonary surfactant | 52 | 95 | 0.537 | 2.28E−01 | Decrease | 95 | 45 | 0.547 | 1.84E−01 | Increase |
| protein A | | | | | | | | | | |
| Pulmonary surfactant | 105 | 182 | 0.505 | 4.47E−01 | Increase | 182 | 63 | 0.529 | 2.53E−01 | Increase |
| protein B | | | | | | | | | | |
| Pulmonary surfactant | 112 | 194 | 0.545 | 9.20E−02 | Increase | 194 | 63 | 0.519 | 3.22E−01 | Decrease |
| protein D | | | | | | | | | | |
| PTEN | 113 | 191 | 0.624 | <1.0E−03 | Increase | 191 | 65 | 0.515 | 3.63E−01 | Decrease |
| RAGE | 119 | 199 | 0.582 | 6.88E−03 | Increase | 199 | 67 | 0.531 | 2.20E−01 | Decrease |
| sICAM1 | 20 | 30 | 0.558 | 2.36E−01 | Increase | 30 | 20 | 0.540 | 3.16E−01 | Increase |
| Sphingosine Kinase I | 103 | 173 | 0.562 | 4.02E−02 | Increase | 173 | 58 | 0.556 | 9.41E−02 | Decrease |
| Tissue Factor | 22 | 33 | 0.534 | 3.35E−01 | Decrease | 33 | 22 | 0.507 | 4.65E−01 | Decrease |
| TNF-a | 22 | 33 | 0.501 | 4.94E−01 | Increase | 33 | 22 | 0.508 | 4.59E−01 | Increase |
| TNFR1a | 105 | 182 | 0.613 | <1.0E−03 | Increase | 182 | 64 | 0.692 | <1.0E−03 | Increase |
| sTNFR14 (LIGHT) | 40 | 85 | 0.551 | 1.80E−01 | Decrease | 85 | 44 | 0.505 | 4.60E−01 | Increase |
| sTREM-1 | 74 | 117 | 0.593 | 1.52E−02 | Increase | 117 | 38 | 0.703 | <1.0E−03 | Increase |
| TREM-1sv | 22 | 33 | 0.576 | 1.69E−01 | Decrease | 33 | 21 | 0.557 | 2.35E−01 | Increase |
| uPAR | 80 | 152 | 0.537 | 1.77E−01 | Increase | 152 | 58 | 0.707 | <1.0E−03 | Increase |
| VCAM-1 | 38 | 80 | 0.591 | 6.17E−02 | Increase | 80 | 46 | 0.590 | 4.03E−02 | Decrease |

| | Univariate ROC area | | | | |
|---|---|---|---|---|---|
| | “control” | “disease” | | | |
| | N (Alive | N (Dead | | | |
| | within 30 | at 30 | | | Change with |
| | days) | days) | ROC area | p-value | disease |
| Adiponectin | 91 | 9 | 0.722 | 1.79E−02 | Increase |
| Adrenomedullin | 139 | 15 | 0.638 | 4.13E−02 | Increase |
| Angiotensinogen | 48 | 6 | 0.677 | 6.04E−02 | Increase |
| Apolipoprotein C1 | 59 | 9 | 0.539 | 3.36E−01 | Decrease |
| Big Endothelin-1 | 192 | 26 | 0.589 | 6.06E−02 | Increase |
| BNP₁₋₁₀₈ | 28 | 7 | 0.633 | 8.81E−02 | Increase |
| BNP₇₉₋₁₀₈ | 28 | 7 | 0.526 | 4.01E−01 | Increase |
| BNP (BNP₇₇₋₁₀₈) | 131 | 16 | 0.662 | 8.10E−03 | Increase |
| BNP₃₋₁₀₈ | 126 | 17 | 0.559 | 2.27E−01 | Increase |
| Complement C3a | 62 | 9 | 0.543 | 2.97E−01 | Decrease |
| Calcitonin | 131 | 17 | 0.547 | 2.44E−01 | Increase |
| Caspase-3 | 128 | 15 | 0.530 | 3.68E−01 | Decrease |
| CCL16 | 25 | 4 | 0.650 | 8.57E−02 | Increase |
| CCL19 | 131 | 17 | 0.587 | 1.83E−01 | Increase |
| CCL20 | 145 | 17 | 0.714 | <1.0E−03 | Increase |
| CCL23 | 122 | 15 | 0.617 | 1.01E−01 | Increase |
| CCL26 | 59 | 9 | 0.552 | 3.17E−01 | Decrease |
| CCL4 (MIP1β) | 141 | 16 | 0.554 | 2.40E−01 | Increase |
| CCL5 | 101 | 14 | 0.565 | 2.37E−01 | Decrease |
| CCL8 | 124 | 14 | 0.554 | 2.65E−01 | Increase |
| CK-BB | 133 | 15 | 0.546 | 2.93E−01 | Decrease |
| CK-MB | 190 | 24 | 0.617 | 3.84E−02 | Increase |
| C-reactive protein (CRP) | 133 | 16 | 0.592 | 1.28E−01 | Increase |
| CXCL5 | 59 | 9 | 0.599 | 2.08E−01 | Decrease |
| CXCL9 | 37 | 8 | 0.649 | 4.37E−02 | Decrease |
| CXCL13 | 125 | 15 | 0.652 | 4.18E−02 | Increase |
| CXCL16 | 103 | 14 | 0.684 | 1.50E−02 | Increase |
| CXCL6 | 143 | 16 | 0.558 | 2.52E−01 | Increase |
| Cystatin C | 194 | 24 | 0.718 | 2.75E−05 | Increase |
| D-Dimer | 134 | 16 | 0.703 | 1.20E−03 | Increase |
| sDR6 | 145 | 17 | 0.569 | 1.71E−01 | Increase |
| Glutathione-S-transferase | 139 | 17 | 0.571 | 2.03E−01 | Increase |
| A (GSTA) | | | | | |
| HSP-60 | 91 | 9 | 0.775 | <1.0E−03 | Increase |
| HMG-1 | 130 | 17 | 0.638 | 4.16E−02 | Increase |
| I-FABP | 31 | 7 | 0.537 | 3.82E−01 | Increase |
| IGFBP-1 | 117 | 16 | 0.612 | 6.50E−02 | Increase |
| IL-10 | 140 | 16 | 0.542 | 2.78E−01 | Increase |
| IL-1β | 46 | 6 | 0.612 | 2.01E−01 | Increase |
| IL-1ra | 134 | 16 | 0.702 | <1.0E−03 | Increase |
| IL-22 | 129 | 17 | 0.583 | 1.25E−01 | Increase |
| IL2sRA | 120 | 14 | 0.705 | 3.93E−03 | Increase |
| IL-6 | 126 | 17 | 0.550 | 2.68E−01 | Increase |
| IL-8 | 133 | 16 | 0.624 | 3.39E−02 | Decrease |
| MCP-1 | 61 | 9 | 0.581 | 2.16E−01 | Increase |
| MIF | 139 | 17 | 0.607 | 4.23E−02 | Increase |
| MMP9 | 129 | 17 | 0.594 | 8.62E−02 | Decrease |
| MPO | 134 | 16 | 0.683 | 3.62E−03 | Increase |
| Myoglobin | 131 | 16 | 0.641 | 4.45E−02 | Increase |
| NGAL | 195 | 24 | 0.528 | 3.32E−01 | Decrease |
| PAI-1 | 126 | 15 | 0.639 | 4.85E−02 | Increase |
| PLGF-1 | 192 | 26 | 0.577 | 8.97E−02 | Decrease |
| PLGF-1 + PLGF-2 | 127 | 14 | 0.727 | <1.0E−03 | Increase |
| Protein C Activated | 73 | 9 | 0.556 | 2.40E−01 | Increase |
| Protein C Total | 132 | 17 | 0.611 | 7.87E−02 | Decrease |
| Pulmonary surfactant | 61 | 9 | 0.653 | 2.46E−02 | Increase |
| protein A | | | | | |
| Pulmonary surfactant | 145 | 17 | 0.672 | 5.39E−03 | Increase |
| protein B | | | | | |
| Pulmonary surfactant | 128 | 15 | 0.593 | 1.13E−01 | Increase |
| protein D | | | | | |
| PTEN | 125 | 16 | 0.504 | 4.78E−01 | Increase |
| RAGE | 133 | 16 | 0.576 | 1.43E−01 | Increase |
| sICAM1 | 25 | 7 | 0.623 | 1.71E−01 | Increase |
| Sphingosine Kinase I | 141 | 17 | 0.626 | 4.52E−02 | Decrease |
| TIMP-1 | 62 | 5 | 0.540 | 3.78E−01 | Increase |
| Tissue Factor | 31 | 7 | 0.588 | 2.08E−01 | Decrease |
| TNF-a | 31 | 7 | 0.516 | 4.34E−01 | Decrease |
| TNFR1a | 145 | 17 | 0.746 | <1.0E−03 | Increase |
| sTNFRSF3 (Lymphotoxin | 122 | 16 | 0.757 | <1.0E−03 | Increase |
| B Receptor) | | | | | |
| sTNFRSF7 (CD27) | 91 | 9 | 0.762 | <1.0E−03 | Increase |
| sTNFRSF11A (RANK) | 191 | 24 | 0.700 | <1.0E−03 | Increase |
| sTNFSF14 (LIGHT) | 48 | 6 | 0.708 | 6.12E−02 | Increase |
| sTREM-1 | 114 | 15 | 0.754 | <1.0E−03 | Increase |
| TREM-1sv | 31 | 6 | 0.519 | 4.33E−01 | Increase |
| UCRP | 91 | 9 | 0.667 | 4.89E−02 | Increase |
| uPAR | 120 | 14 | 0.723 | 3.59E−03 | Increase |
| VCAM-1 | 42 | 6 | 0.532 | 3.77E−01 | Increase |

For peptidoglycan recognition protein, an assay was developed having a minimum detectable level of 0.81 ng/mL and a maximum level of 400 ng/mL. In the following data, SIRS/Sepsis refers to subjects for which a diagnosis of SIRS was made, but for which sepsis could not be unequivocally demonstrated. The category “Severe Sepsis and/or Shock at >0” refers to subjects that did not have either severe sepsis or septic shock at the time of presentation for medical care, but who progressed to a diagnosis of Severe Sepsis and/or Shock. This contrasts with the “Severe Sepsis and/or Shock” category, which refers to subjects presenting for medical care with either severe sepsis or septic shock. All samples measured were at the time of presentation of the subject.

| | | | | | Severe | |
|---|---|---|---|---|---|---|
| | | | | | Sepsis | Severe |
| | | | | | and/or | Sepsis |
| | | | SIRS/ | | Shock | and/or |
| | Normal | SIRS | Sepsis | Sepsis | at >0 | Shock |
| N | 173 | 81 | 115 | 101 | 99 | 176 |
| Concentration | 48.44 | 58.33 | 65.55 | 116.37 | 117.22 | 135.68 |
| (5th percentile) | | | | | | |
| Concentration | 48.44 | 58.33 | 65.55 | 116.37 | 117.22 | 135.68 |
| (25th | | | | | | |
| percentile) | | | | | | |
| Concentration | 64.81 | 88.66 | 106.82 | 209.02 | 209.15 | 346.14 |
| (50th | | | | | | |
| percentile) | | | | | | |
| Concentration | 86.65 | 127.33 | 204.46 | 400.00 | 400.00 | 400.00 |
| (75th | | | | | | |
| percentile) | | | | | | |
| Concentration | 172.44 | 372.94 | 400.00 | 400.00 | 400.00 | 400.00 |
| (95th | | | | | | |
| percentile) | | | | | | |

The ability of peptidoglycan recognition protein to diagnose sepsis and to differentiate causes of sepsis was calculated using standard ROC analysis. The results are summarized in the following table:

| | N (1^{st} | N (2^{nd} | ROC | |
|---|---|---|---|---|
| Groups analyzed | group) | group) | area | p |
| SIRS vs. All Sepsis (Sepsis + Severe | 81 | 376 | 0.800 | <0.0001 |
| Sepsis and/or Shock at any time) | | | | |
| Sepsis vs. Severe Sepsis and/or | 200 | 176 | 0.578 | 0.0046 |
| Shock at 0 hr | | | | |
| SIRS, SIRS/Sepsis and Sepsis vs. | 297 | 99 | 0.654 | <0.0001 |
| Severe Sepsis and/or Shock at >0 hr | | | | |
| Alive vs. Dead at Day 3 | 659 | 20 | 0.621 | 0.0394 |
| Alive vs. Dead at Day 30 | 494 | 57 | 0.604 | 0.0047 |
| Normal vs. SIRS | 173 | 81 | 0.659 | <0.0001 |
| Normal vs. All Sepsis | 173 | 376 | 0.893 | <0.0001 |

For carboxypeptidase B, an assay was developed that detected procarboxypeptidase B but not active carboxypeptidase B by having one antibody in a sandwich assay that binds to the activation peptide. This assay exhibited a minimum detectable level of 0.1 ng/mL and a maximum level of 200 ng/mL. In the following data, SIRS/Sepsis refers to subjects for which a diagnosis of SIRS was made, but for which sepsis could not be unequivocally demonstrated. The category “Severe Sepsis and/or Shock at >0” refers to subjects that did not have either severe sepsis or septic shock at the time of presentation for medical care, but who progressed to a diagnosis of Severe Sepsis and/or Shock, This contrasts with the “Severe Sepsis and/or Shock” category, which refers to subjects presenting for medical care with either severe sepsis or septic shock. All samples measured were at the time of presentation of the subject.

| | | | | | Severe | |
|---|---|---|---|---|---|---|
| | | | | | Sepsis | Severe |
| | | | | | and/or | Sepsis |
| | | | SIRS/ | | Shock | and/or |
| | Normal | SIRS | Sepsis | Sepsis | at >0 | Shock |
| N | 243 | 83 | 118 | 104 | 100 | 177 |
| Concentration | 3.14 | 2.72 | 1.88 | 3.21 | 2.33 | 4.56 |
| (5th percentile) | | | | | | |
| Concentration | 3.14 | 2.72 | 1.88 | 3.21 | 2.33 | 4.56 |
| (25th percentile) | | | | | | |
| Concentration | 6.09 | 5.54 | 5.44 | 7.75 | 8.27 | 10.05 |
| (50th percentile) | | | | | | |
| Concentration | 12.70 | 11.53 | 11.29 | 17.67 | 28.43 | 32.56 |
| (75th percentile) | | | | | | |
| Concentration | 39.74 | 56.10 | 37.89 | 43.71 | 98.94 | 129.01 |
| (95th percentile) | | | | | | |

The ability of procarboxypeptidase B to diagnose sepsis and to differentiate causes of sepsis was calculated using standard ROC analysis. The results are summarized in the following table:

| | N (1^{st} | N (2^{nd} | ROC | |
|---|---|---|---|---|
| Groups analyzed | group) | group) | area | p |
| SIRS vs. All Sepsis (Sepsis + Severe | 83 | 381 | 0.596 | 0.0015 |
| Sepsis and/or Shock at any time) | | | | |
| Sepsis vs. Severe Sepsis and/or | 204 | 177 | 0.558 | 0.0243 |
| Shock at 0 hr | | | | |
| SIRS, SIRS/Sepsis and Sepsis vs. | 305 | 100 | 0.561 | 0.0468 |
| Severe Sepsis and/or Shock at >0 hr | | | | |
| Alive vs. Dead at Day 3 | 682 | 20 | 0.530 | 0.3306 |
| Alive vs. Dead at Day 30 | 517 | 55 | 0.619 | 0.0021 |
| Normal vs. SIRS | 243 | 83 | 0.522 | 0.2800 |
| Normal vs. All Sepsis | 243 | 381 | 0.579 | 0.0002 |

For alanine aminotransferase, an assay was developed having a minimum detectable level of 2.21 ng/mL and a maximum level of 1000 ng/mL. In the following data, SIRS/Sepsis refers to subjects for which a diagnosis of SIRS was made, but for which sepsis could not be unequivocally demonstrated. The category “Severe Sepsis and/or Shock at >0” refers to subjects that did not have either severe sepsis or septic shock at the time of presentation for medical care, but who progressed to a diagnosis of Severe Sepsis and/or Shock, This contrasts with the “Severe Sepsis and/or Shock” category, which refers to subjects presenting for medical care with either severe sepsis or septic shock. All samples measured were at the time of presentation of the subject.

| | | | | | Severe | |
|---|---|---|---|---|---|---|
| | | | | | Sepsis | Severe |
| | | | | | and/or | Sepsis |
| | | | SIRS/ | | Shock | and/or |
| | Normal | SIRS | Sepsis | Sepsis | at >0 | Shock |
| N | 174 | 81 | 115 | 101 | 99 | 175 |
| Concentration | 80.8 | 103.3 | 86.5 | 86.8 | 76.4 | 78.5 |
| (5th percentile) | | | | | | |
| Concentration | 80.8 | 103.3 | 86.5 | 86.8 | 76.4 | 78.5 |
| (25th percentile) | | | | | | |
| Concentration | 119.7 | 144.4 | 126.7 | 130.0 | 103.7 | 145.1 |
| (50th percentile) | | | | | | |
| Concentration | 177.4 | 232.0 | 205.9 | 198.5 | 179.0 | 293.1 |
| (75th percentile) | | | | | | |
| Concentration | 280.4 | 412.6 | 763.3 | 558.2 | 598.3 | 1000 |
| (95th percentile) | | | | | | |

The ability of peptidoglycan recognition protein to diagnose sepsis and to differentiate causes of sepsis was calculated using standard ROC analysis. The results are summarized in the following table:

| | N (1^{st} | N (2^{nd} | ROC | |
|---|---|---|---|---|
| Groups analyzed | group) | group) | area | p |
| SIRS vs. All Sepsis (Sepsis + Severe | 81 | 375 | 0.55 | 0.04 |
| Sepsis and/or Shock at any time) | | | | |
| Sepsis vs. Severe Sepsis and/or Shock | 200 | 175 | 0.55 | 0.06 |
| at 0 hr | | | | |
| SIRS, SIRS/Sepsis and Sepsis vs. | 297 | 99 | 0.58 | 0.01 |
| Severe Sepsis and/or Shock at >0 hr | | | | |
| Alive vs. Dead at Day 3 | 661 | 19 | 0.51 | 0.46 |
| Alive vs. Dead at Day 30 | 496 | 56 | 0.50 | 0.49 |
| Normal vs. SIRS | 174 | 81 | 0.62 | 0.001 |
| Normal vs. All Sepsis | 174 | 375 | 0.54 | 0.06 |

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method of diagnosing SIRS, sepsis, severe sepsis, septic shock, or MODS in a subject, or assigning a prognostic risk for one or more clinical outcomes for a subject suffering from SIRS, sepsis, severe sepsis, septic shock, or MODS, the method comprising:
performing an assay method on one or more samples obtained from said subject, wherein said assay method comprises performing a plurality of immunoassays, provided that at least two of said plurality of immunoassays detect markers selected from the group consisting of NT-proBNP, proBNP, BNP₇₉₋₁₀₈, BNP, BNP₃₋₁₀₈, CCL19, CCL23, CRP, cystatin C, D-dimer, IL-1ra, IL-2sRa, myeloperoxidase, myoglobin, NGAL, lymphotoxin B receptor, peptidoglycan recognition protein, procalcitonin, procarboxypeptidase B, active protein C, latent protein C, total protein C, and sTNFR1a; and
relating the immunoassay results obtained from said assay method to one or more diagnoses or prognoses selected from the group consisting of the presence or absence of SIRS, the presence or absence of sepsis, the presence or absence of severe sepsis, the presence or absence of septic shock, and the prognostic risk of one or more clinical outcomes for the subject suffering from or believed to suffer from SIRS, sepsis, severe sepsis, septic shock, or MODS.

2. A method according to claim 1, wherein said assay method comprises performing at least two immunoassays that detect markers selected from the group consisting of NT-proBNP, proBNP, BNP₇₉₋₁₀₈, BNP, BNP₃₋₁₀₈, CCL23, CRP, D-dimer, IL-1ra, NGAL, peptidoglycan recognition protein, active protein C, latent protein C, total protein C, and sTNFR1a.

3. A method according to claim 1, wherein said assay method comprises performing at least three immunoassays that detect markers selected from the group consisting of NT-proBNP, proBNP, BNP₇₉₋₁₀₈, BNP, BNP₃₋₁₀₈, CCL23, CRP, D-dimer, IL-1ra, NGAL, peptidoglycan recognition protein, active protein C, latent protein C, total protein C, and sTNFR1a.

4. A method according to claim 1, wherein said assay method comprises performing at least four immunoassays that detect markers selected from the group consisting of NT-proBNP, proBNP, BNP₇₉₋₁₀₈, BNP, BNP₃₋₁₀₈, CCL23, CRP, D-dimer, IL-1ra, NGAL, peptidoglycan recognition protein, active protein C, latent protein C, total protein C, and sTNFR1a.

5. A method according to claim 1, wherein said assay method comprises performing at least five immunoassays that detect markers selected from the group consisting of NT-proBNP, proBNP, BNP₇₉₋₁₀₈, BNP, BNP₃₋₁₀₈, CCL23, CRP, D-dimer, IL-1ra, NGAL, peptidoglycan recognition protein, active protein C, latent protein C, total protein C, and sTNFR1a.

6. A method according to claim 1, wherein the assay method further comprises performing one or more additional immunoassays that detect one or more additional markers other than those listed in claim 1.

7. A method according to claim 1, wherein said method provides a ROC area of at least 0.7 for the diagnosis of sepsis or for the prognostic risk of mortality.

8. A method according to claim 1, wherein the method comprises performing an immunoassay that detects one or more of BNP, proBNP, NT-proBNP, or BNP₃₋₁₀₈.

9. A method according to claim 1, wherein the method comprises performing an immunoassay that detects C-reactive protein.

10. A method according to claim 1, wherein the method comprises performing an immunoassay that detects CCL23.

11. A method according to claim 1, wherein the method comprises performing an immunoassay that detects D-dimer.

12. A method according to claim 1, wherein the method comprises performing an immunoassay that detects NGAL.

13. A method according to claim 1, wherein the method comprises performing an immunoassay that detects one or more of active protein C, latent protein C, total protein C.

14. A method according to claim 1, wherein the method comprises performing an immunoassay that detects peptidoglycan recognition protein.

15. A method according to claim 1, wherein the method comprises performing an immunoassay that detects sTNFR1a.

16. A method according to claim 1, wherein the method comprises performing an immunoassay that detects IL-1ra.

17. A method according to claim 1, wherein the sample is from a human.

18. A method according to claim 1, wherein the sample is selected from the group consisting of blood, serum, and plasma.

19. A device for performing the method of claim 1, comprising a plurality of discrete locations on a solid phase, each comprising antibodies for performing said immunoassays.

20. A method according to claim 1, wherein the relating step comprises comparing a result obtained from each immunoassay to a predetermined threshold level selected to indicate the presence or absence of SIRS, the presence or absence of sepsis, the presence or absence of severe sepsis, the presence or absence of septic shock, or the prognostic risk of one or more clinical outcomes for the subject suffering from or believed to suffer from SIRS, sepsis, severe sepsis, septic shock, or MODS.

21. A method according to claim 1, wherein the relating step comprises comparing a single result to a predetermined threshold level selected to indicate the presence or absence of SIRS, the presence or absence of sepsis, the presence or absence of severe sepsis, the presence or absence of septic shock, or the prognostic risk of one or more clinical outcomes for the subject suffering from or believed to suffer from SIRS, sepsis, severe sepsis, septic shock, or MODS, wherein said single result is a function of each immunoassay result obtained from said assay method.

22. A method according to claim 1, wherein the relating step comprises relating both the immunoassay results obtained from said assay method, and one or more variables that are not immunoassay results, to one or more diagnoses or prognoses selected from the group consisting of the presence or absence of SIRS, the presence or absence of sepsis, the presence or absence of severe sepsis, the presence or absence of septic shock, and the prognostic risk of one or more clinical outcomes for the subject suffering from or believed to suffer from SIRS, sepsis, severe sepsis, septic shock, or MODS.

23. A method according to claim 22, wherein the variables that are not immunoassay results comprise one or more of heart rate, temperature, respiration rate, white blood cell count, blood gas level, venous blood pH, blood lactate level, renal function, electrolyte level, blood pressure, pulmonary wedge pressure, or blood culture result.

24. A method according to claim 1, wherein the method comprises performing an immunoassay that detects one or more of BNP, proBNP, NT-proBNP, or BNP₃₋₁₀₈, an immunoassay that detects one or more of active protein C, latent protein C, total protein C, and at least one immunoassay that detects a marker selected from the group consisting of CCL23, CRP, D-dimer, IL-1ra, NGAL, peptidoglycan recognition protein, and sTNFR1a.

25. A method according to claim 1, wherein the method comprises performing an immunoassay that detects one or more of BNP, proBNP, NT-proBNP, or BNP₃₋₁₀₈, at least one immunoassay that detects a marker selected from the group consisting of C-reactive protein, D-dimer, and IL-1ra, and at least one immunoassay that detects a marker selected from the group consisting of CCL23, peptidoglycan recognition protein, and sTNFR1a.

26. A method according to claim 1, wherein the method comprises performing an immunoassay that detects peptidoglycan recognition protein and an immunoassay that detects sTNFR1a.

27. A method according to claim 1, wherein the method comprises performing an immunoassay that detects one or more of BNP, proBNP, NT-proBNP, or BNP₃₋₁₀₈, and at least one immunoassay that detects a marker selected from the group consisting of CCL19, CCL23, CRP, cystatin C, D-dimer, IL-1ra, IL-2sRa, myeloperoxidase, myoglobin, NGAL, lymphotoxin B receptor, peptidoglycan recognition protein, procalcitonin, procarboxypeptidase B, active protein C, latent protein C, total protein C, and sTNFR1a.

28. A method of diagnosing SIRS in a subject, differentiating causes of SIRS in a subject, or assigning a prognostic risk of one or more future clinical outcomes to a subject suffering from SIRS, sepsis, severe sepsis, septic shock, or MODS, the method comprising:
performing assays configured to detect two or more markers selected from the group consisting of alanine aminotransferase, NT-proBNP, proBNP, BNP₇₉₋₁₀₈, BNP, BNP₃₋₁₀₈, CCL19, CRP, cystatin C, D-dimer, IL-2sRa, myeloperoxidase, myoglobin, NGAL, lymphotoxin B receptor, peptidoglycan recognition protein, procalcitonin, procarboxypeptidase B, active protein C, latent protein C, total protein C, and TNFR1a on one or more samples obtained from said subject; and
correlating the results of said assays to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

29. A method according to claim 28, wherein the method comprises performing assays configured to detect one or more markers selected from the group consisting of alanine aminotransferase, lymphotoxin B receptor, peptidoglycan recognition protein, and procarboxypeptidase B.

30. A method according to claim 28, wherein the method comprises performing assays configured to detect two or more markers selected from the group consisting of lymphotoxin B receptor, peptidoglycan recognition protein, and procarboxypeptidase B.

31. A method according to claim 28, wherein the method comprises performing assays configured to detect two or more of alanine aminotransferase, BNP, CRP, cystatin C, D-dimer, IL-2sRa, NGAL, lymphotoxin B receptor, peptidoglycan recognition protein, procalcitonin, procarboxypeptidase B, total protein C, and TNFR1a, wherein said assay configured to detect BNP is optionally replaced with an assay configured to detect BNP₃₋₁₀₈, NT-proBNP, proBNP, or BNP₇₉₋₁₀₈, and wherein said assay configured to detect total protein C is optionally replaced with an assay configured to detect active protein C or latent protein C.

32. A method according to claim 28, wherein the method comprises performing assays configured to detect three or more of alanine aminotransferase, BNP, CRP, cystatin C, D-dimer, IL-2sRa, NGAL, lymphotoxin B receptor, peptidoglycan recognition protein, procalcitonin, procarboxypeptidase B, total protein C, and TNFR1a, wherein said assay configured to detect BNP is optionally replaced with an assay configured to detect BNP₃₋₁₀₈, NT-proBNP, proBNP, or BNP₇₉₋₁₀₈, and wherein said assay configured to detect total protein C is optionally replaced with an assay configured to detect active protein C or latent protein C.

33. A method according to claim 28, wherein the method comprises performing assays configured to detect four or more of alanine aminotransferase, BNP, CRP, cystatin C, D-dimer, IL-2sRa, NGAL, lymphotoxin B receptor, peptidoglycan recognition protein, procalcitonin, procarboxypeptidase B, total protein C, and TNFR1a, wherein said assay configured to detect BNP is optionally replaced with an assay configured to detect BNP₃₋₁₀₈, NT-proBNP, proBNP, or BNP₇₉₋₁₀₈, and wherein said assay configured to detect total protein C is optionally replaced with an assay configured to detect active protein C or latent protein C.

34. A method according to claim 28, wherein the method comprises performing assays configured to detect five or more of alanine aminotransferase, BNP, CRP, cystatin C, D-dimer, IL-2sRa, NGAL, lymphotoxin B receptor, peptidoglycan recognition protein, procalcitonin, procarboxypeptidase B, total protein C, and TNFR1a, wherein said assay configured to detect BNP is optionally replaced with an assay configured to detect BNP₃₋₁₀₈, NT-proBNP, proBNP, or BNP₇₉₋₁₀₈, and wherein said assay configured to detect total protein C is optionally replaced with an assay configured to detect active protein C or latent protein C.

35. A method according to claim 28, wherein the method comprises performing assays configured to detect two or more markers selected from the group consisting of alanine aminotransferase, BNP, BNP₃₋₁₀₈, NT-proBNP, proBNP, BNP₇₉₋₁₀₈, cystatin C, D-dimer, IL-2sRa, NGAL, lymphotoxin B receptor, peptidoglycan recognition protein, procalcitonin, procarboxypeptidase B, D-dimer, total protein C, active protein C, and latent protein C.

36. A method according to claim 28, wherein the method comprises performing assays configured to detect three or more markers selected from the group consisting of alanine aminotransferase, BNP, BNP₃₋₁₀₈, NT-proBNP, proBNP, BNP₇₉₋₁₀₈, cystatin C, D-dimer, IL-2sRa, NGAL, lymphotoxin B receptor, peptidoglycan recognition protein, procalcitonin, procarboxypeptidase B, D-dimer, total protein C, active protein C, and latent protein C.

37. A method according to claim 28, wherein the method comprises performing assays configured to detect four or more markers selected from the group consisting of alanine aminotransferase, BNP, BNP₃₋₁₀₈, NT-proBNP, proBNP, BNP₇₉₋₁₀₈, cystatin C, D-dimer, IL-2sRa, NGAL, lymphotoxin B receptor, peptidoglycan recognition protein, procalcitonin, procarboxypeptidase B, D-dimer, total protein C, active protein C, and latent protein C.

38. A method according to claim 28, wherein the method comprises performing assays configured to detect five or more markers selected from the group consisting of alanine aminotransferase, BNP, BNP₃₋₁₀₈, NT-proBNP, proBNP, BNP₇₉₋₁₀₈, cystatin C, D-dimer, IL-2sRa, NGAL, lymphotoxin B receptor, peptidoglycan recognition protein, procalcitonin, procarboxypeptidase B, D-dimer, total protein C, active protein C, and latent protein C.

39. A method according to one of claims 28-38, wherein the method comprises performing one or more additional assays configured to detect one or more markers in addition to markers selected from the group consisting of alanine aminotransferase, NT-proBNP, proBNP, BNP₇₉₋₁₀₈, BNP, BNP₃₋₁₀₈, CCL19, CRP, cystatin C, D-dimer, IL-2sRa, myeloperoxidase, myoglobin, NGAL, lymphotoxin B receptor, peptidoglycan recognition protein, procalcitonin, procarboxypeptidase B, active protein C, latent protein C, total protein C, and TNFR1a; and
wherein said correlating step comprises correlating the results of said assays and the results of said additional assay(s) to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

40. A method according to claim 39, wherein the assay configured to detect BNP also detects one or more of BNP₃₋₁₀₈, NT-proBNP, proBNP, and BNP₇₉₋₁₀₈.

41. A method of diagnosing SIRS in a subject, differentiating causes of SIRS in a subject, or assigning a prognostic risk of one or more future clinical outcomes to a subject suffering from SIRS, sepsis, severe sepsis, septic shock, or MODS, the method comprising:
performing one or more assays configured to detect one or more markers selected from the group consisting of adiponectin, angiotensinogen, apolipoprotein C1, CCL20, CXCL5, CXCL9, L-FABP, NGAL, peptidoglycan recognition protein, procarboxypeptidase B, placental growth factor-1, placental growth factor-2, sTNFRSF3, sTNFRSF7, and UCRP;
correlating the assay result(s) to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

42. A method according to claim 41, wherein said method comprises performing one or more additional assays configured to detect one or more markers selected from the group consisting of alanine aminotransferase, adrenomedullin, big endothelin-1, NT-proBNP, proBNP, BNP₇₉₋₁₀₈, BNP, BNP₃₋₁₀₈, complement C3a, calcitonin, caspase-3, CCL19, CCL23, CCL26, CCL4, CCL5, CCL8, creatine kinase-BB, C-reactive protein, CXCL13, CXCL16, CXCL6, cystatin C, D-Dimer, sDR6, glutathione-S-transferase A, HMG-1, intestinal fatty acid binding protein, IGFBP-1, IL-10, IL-1β, IL-1RA, IL-22, IL-2sRa, IL-6, IL-8, MCP-1, macrophage migration inhibitory factor, matrix metalloproteinase 9, myeloperoxidase, myoglobin, PAI-1, procalcitonin, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TIMP-1, TNF-α, TNF-R1a, TNF-sR14, sTNFRSF11A, sTREM-1, TREM-1sv, uPAR, and VCAM-1 on a blood, serum, or plasma sample obtained from said subject, to generate one or more assay results; and
wherein said correlating step comprises correlating the result(s) of said assays and the results of said additional assay(s) to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

43. A method according to claim 41, wherein said method comprises performing assays configured to detect two or more markers selected from the group consisting of angiotensinogen, apolipoprotein C1, CCL20, CXCL5, CXCL9, L-FABP, NGAL, peptidoglycan recognition protein, procarboxypeptidase B, placental growth factor-1, placental growth factor-2, sTNFRSF3, sTNFRSF7, and UCRP, or their biosynthetic precursors.

44. A method according to claim 41, wherein the method of differentiating causes of SIRS differentiates between sepsis and severe sepsis or septic shock.

45. A method according to claim 41, wherein the method of differentiating causes of SIRS differentiates between sepsis or severe sepsis and septic shock.

46. A method according to claim 42, wherein the one or more additional markers are selected from the group consisting of markers related to blood pressure regulation, markers related to inflammation, markers related to apoptosis, and markers related to coagulation and hemostasis.

47. A method according to claim 41, wherein the subject is a human.

48. A method according to claim 41, wherein the assay is an immunoassay.

49. A method according to claim 45, wherein said one or more additional assays comprise one or more additional assays configured to detect one or more markers selected from the group consisting of alanine aminotransferase, NT-proBNP, proBNP, BNP₇₉₋₁₀₈, BNP, BNP₃₋₁₀₈, CRP, cystatin C, D-dimer, IL-2sRa, NGAL, lymphotoxin B receptor, procalcitonin, active protein C, latent protein C, total protein C, and TNFR1a.

50. A method according to claim 41, wherein the method provides a prognostic risk of mortality.

51. A method according to claim 42, wherein the method comprises performing assays configured to detect one or more of BNP, NT-proBNP, proBNP, BNP₃₋₁₀₈, or BNP_{79-108.}

52. A method according to claim 42, wherein the method comprises performing an assay configured to detect BNP, NT-proBNP, proBNP, BNP₃₋₁₀₈, or BNP₇₉₋₁₀₈.

53. A method according to claim 52, wherein the assay configured to detect BNP also detects one or more of BNP₃₋₁₀₈, NT-proBNP, proBNP, and BNP₇₉₋₁₀₈.

54. A method of diagnosing SIRS in a subject, differentiating causes of SIRS in a subject, or assigning a prognostic risk of one or more future clinical outcomes to a subject suffering from SIRS, sepsis, severe sepsis, septic shock, or MODS, the method comprising:
performing one or more assays configured to detect two or more markers selected from the group consisting of NT-proBNP, proBNP, BNP₇₉₋₁₀₈, BNP, BNP₃₋₁₀₈, CCL19, D-dimer, myeloperoxidase, myoglobin, active protein C, latent protein C, and total protein C on one or more samples obtained from said subject to generate one or more assay results; and
correlating the assay results to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

55. A method according to claim 54, wherein the method comprises performing assays configured to detect two or more markers selected from the group consisting of BNP, CCL19, D-dimer, myeloperoxidase, myoglobin, and total protein C.

56. A method according to claim 54, wherein the method comprises performing assays configured to detect three or more markers selected from the group consisting of BNP, CCL19, D-dimer, myeloperoxidase, myoglobin, and total protein C.

57. A method according to claim 54, wherein the method comprises performing assays configured to detect four or more markers selected from the group consisting of BNP, CCL19, D-dimer, myeloperoxidase, myoglobin, and total protein C.

58. A method according to claim 54, wherein the method comprises performing assays configured to detect five or more markers selected from the group consisting of BNP, CCL19, D-dimer, myeloperoxidase, myoglobin, and total protein C.

59. A method according to claim 54, wherein the method comprises performing assays configured to detect each of the markers selected from the group consisting of BNP, CCL19, D-dimer, myeloperoxidase, myoglobin, and total protein C.

60. A method according to one of claims 54-59, wherein the method comprises performing assays configured to detect one or more markers in addition to marker(s) selected from the group consisting of BNP, CCL19, D-dimer, myeloperoxidase, myoglobin, and total protein C.

61. A method of diagnosing SIRS in a subject, differentiating causes of SIRS in a subject, or assigning a prognostic risk of one or more future clinical outcomes to a subject suffering from SIRS, sepsis, severe sepsis, septic shock, or MODS, the method comprising:
measuring the presence or amount of two or more markers selected from the group consisting of BNP, CCL19, D-dimer, myeloperoxidase, myoglobin, and total protein C, or markers related thereto, on one or more samples obtained from said subject to generate one or more assay results; and
correlating the assay results to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

62. A method according to claim 61, wherein the method comprises measuring the presence or amount of three or more markers selected from the group consisting of BNP, CCL19, D-dimer, myeloperoxidase, myoglobin, and total protein C, or markers related thereto.

63. A method according to claim 61, wherein the method comprises measuring the presence or amount of four or more markers selected from the group consisting of BNP, CCL19, D-dimer, myeloperoxidase, myoglobin, and total protein C, or markers related thereto.

64. A method according to claim 61, wherein the method comprises measuring the presence or amount of five or more markers selected from the group consisting of BNP, CCL19, D-dimer, myeloperoxidase, myoglobin, and total protein C, or markers related thereto.

65. A method according to claim 61, wherein the method comprises measuring the presence or amount of each of the markers selected from the group consisting of BNP, CCL19, D-dimer, myeloperoxidase, myoglobin, and total protein C, or markers related thereto.

66. A method according to one of claims 61-65, wherein the method comprises measuring the presence or amount of one or more markers in addition to marker(s) selected from the group consisting of BNP, CCL19, D-dimer, myeloperoxidase, myoglobin, and total protein C, or markers related thereto.

67. A method of diagnosing SIRS in a subject, differentiating causes of SIRS in a subject, or assigning a prognostic risk of one or more future clinical outcomes to a subject suffering from SIRS, sepsis, severe sepsis, septic shock, or MODS, the method comprising:
performing one or more assays configured to detect one or more markers selected from the group consisting of adrenomedullin, angiotensinogen, apolipoprotein C1, big endothelin-1, NT-proBNP, proBNP, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL13, CXCL16, CXCL6, CXCL5, CXCL9, cystatin C, D-Dimer, sDR6, glutathione-S-transferase A, HMG-1, intestinal fatty acid binding protein, IGFBP-1, IL-10, IL-1β, IL-1RA, IL-22, IL-2sRa, IL-6, IL-8, L-FABP, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TIMP-1, TNF-α, TNF-R1a, TNF-sR14, sTNFRSF3, sTNFRSF7, sTNFRSF11A, sTREM-1, TREM-1sv, uPAR, UCRP, and VCAM-1, or their biosynthetic precursors, on a blood, serum, or plasma sample obtained from said subject, to generate one or more assay results; and
correlating the assay result(s) to the presence or absence of SIRS in the subject, or to the presence or absence of sepsis, severe sepsis, septic shock, or MODS in the subject, or to the prognostic risk of one or more clinical outcomes for the subject suffering from or believed to suffer from SIRS, sepsis, severe sepsis, septic shock, or MODS.

68. A method according to claim 67, wherein said method comprises performing one or more assays configured to detect one or more markers selected from the group consisting of angiotensinogen, apolipoprotein C1, CCL20, CXCL5, CXCL9, L-FABP, NGAL, placental growth factor, sTNFRSF3, sTNFRSF7, and UCRP, or their biosynthetic precursors.

69. A method according to claim 67, wherein the method of differentiating causes of SIRS differentiates between sepsis and severe sepsis or septic shock.

70. A method according to claim 67, wherein the method of differentiating causes of SIRS differentiates between sepsis or severe sepsis and septic shock.

71. A method according to claim 67, wherein the method comprises performing one or more assays configured to detect one or more additional markers on a blood, serum, or plasma sample obtained from said subject to generate one or more additional assay results, and wherein the correlating step comprises correlating the assay result and the additional assay result(s) to the presence or absence of SIRS in the subject, or to the presence or absence of sepsis, severe sepsis, septic shock, or MODS in the subject, or to the prognostic risk of one or more clinical outcomes for the subject.

72. A method according to claim 71, wherein the one or more additional markers are selected from the group consisting of markers related to blood pressure regulation, markers related to inflammation, markers related to apoptosis, and markers related to coagulation and hemostasis.

73. A method according to claim 67, wherein the subject is a human.

74. A method according to claim 67, wherein the assay is an immunoassay.

75. A method according to claim 71, wherein said one or more additional markers comprise at least one marker selected from the group consisting of atrial natriuretic factor, C-type natriuretic peptide, lactate, urotensin II, arginine vasopressin, aldosterone, angiotensin I, angiotensin II, angiotensin III, bradykinin, procalcitonin, calcitonin gene related peptide, calcyphosine, creatinine, endothelin-2, endothelin-3, renin, and urodilatin, or their biosynthetic precursors.

76. A method according to claim 71, wherein said one or more additional markers comprise at least one marker selected from the group consisting of LIGHT, CCL16, MMP7, intercellular adhesion molecule-1, intercellular adhesion molecule-2, intercellular adhesion molecule-3, lipocalin-type prostaglandin D synthase, mast cell tryptase, eosinophil cationic protein, KL-6, haptoglobin, tumor necrosis factor β, fibronectin, and vascular endothelial growth factor, or their biosynthetic precursors.

77. A method according to claim 71, wherein said one or more additional markers comprise at least one marker selected from the group consisting of hepcidin, HSP-60, HSP-65, HSP-70, S-FAS ligand, asymmetric dimethylarginine, matrix metalloproteinase 11, matrix metalloproteinase 3, defensin HBD 1, defensin HBD 2, serum amyloid A, oxidized LDL, insulin like growth factor, transforming growth factor β, an inter-α-inhibitor, e-selectin, hypoxia-inducible factor-1α, inducible nitric oxide synthase, intracellular adhesion molecule-1, lactate dehydrogenase, n-acetyl aspartate, prostaglandin E2, and receptor activator of nuclear factor ligand, or their biosynthetic precursors.

78. A method according to claim 71, wherein said one or more additional markers comprise at least one marker selected from the group consisting of plasmin, fibrinogen, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, and thrombus precursor protein, or their biosynthetic precursors.

79. A method according to claim 71, wherein the method comprises performing assays configured to detect two or more of BNP, CCL19, D-dimer, myeloperoxidase, myoglobin, and total protein C, or their biosynthetic precursors.

80. A method according to claim 71, wherein the method comprises performing assays configured to detect three or more of BNP, CCL19, D-dimer, myeloperoxidase, myoglobin, and total protein C, or their biosynthetic precursors.

81. A method according to claim 71, wherein the method comprises performing assays configured to detect four or more of BNP, CCL19, D-dimer, myeloperoxidase, myoglobin, and total protein C, or their biosynthetic precursors.

82. A method according to claim 71, wherein the method comprises performing assays configured to detect BNP, CCL19, D-dimer, myeloperoxidase, myoglobin, and total protein C, or their biosynthetic precursors.

83. A method according to claim 71, wherein the method comprises performing assays configured to detect two or more of BNP, CCL19, D-dimer, myeloperoxidase, myoglobin, and total protein C, or their biosynthetic precursors.

84. A method according to claim 67, wherein the method provides a prognostic risk of mortality.

85. A method according to claim 71, wherein the method comprises performing assays configured to detect one or more of BNP, NT-proBNP, proBNP, BNP₃₋₁₀₈, or BNP₇₉₋₁₀₈.

86. A method according to claim 67, wherein the method comprises performing an assay configured to detect BNP, NT-proBNP, proBNP, BNP₃₋₁₀₈, or BNP₇₉₋₁₀₈.

87. A method according to claim 71, wherein the method comprises performing at least two additional assays configured to detect at least two additional markers on a blood, serum, or plasma sample obtained from said subject to generate at least two additional assay results, and wherein the correlating step comprises correlating the assay result and the additional assay result(s) to the presence or absence of SIRS in the subject, or to the presence or absence of sepsis, severe sepsis, septic shock, or MODS in the subject, or to the prognostic risk of one or more clinical outcomes for the subject.

88. A method according to claim 87, wherein the method comprises performing at least three additional assays configured to detect at least three additional markers on a blood, serum, or plasma sample obtained from said subject to generate at least three additional assay results, and wherein the correlating step comprises correlating the assay result and the additional assay result(s) to the presence or absence of SIRS in the subject, or to the presence or absence of sepsis, severe sepsis, septic shock, or MODS in the subject, or to the prognostic risk of one or more clinical outcomes for the subject.

89. A method according to claim 67, wherein said method comprises performing assays configured to detect at least two markers selected from the group consisting of adrenomedullin, angiotensinogen, apolipoprotein C1, big endothelin-1, NT-proBNP, proBNP, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL13, CXCL16, CXCL6, CXCL5, CXCL9, cystatin C, D-Dimer, sDR6, glutathione-S-transferase A, HMG-1, intestinal fatty acid binding protein, IGFBP-1, IL-10, IL-1β, IL-1RA, IL-22, IL-2sRa, IL-6, IL-8, L-FABP, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TIMP-1, TNF-α, TNF-R1a, TNF-sR14, sTNFRSF3, sTNFRSF7, sTNFRSF11A, sTREM-1, TREM-1sv, uPAR, UCRP, and VCAM-1, or their biosynthetic precursors.

90. A method according to claim 89, wherein said method comprises performing assays configured to detect at least three markers selected from the group consisting of adrenomedullin, angiotensinogen, apolipoprotein C1, big endothelin-1, NT-proBNP, proBNP, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL13, CXCL16, CXCL6, CXCL5, CXCL9, cystatin C, D-Dimer, sDR6, glutathione-S-transferase A, HMG-1, intestinal fatty acid binding protein, IGFBP-1, IL-10, IL-1β, IL-1RA, IL-22, IL-2sRa, IL-6, IL-8, L-FABP, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TIMP-1, TNF-α, TNF-R1a, TNF-sR14, sTNFRSF3, sTNFRSF7, sTNFRSF11A, sTREM-1, TREM-1sv, uPAR, UCRP, and VCAM-1, or their biosynthetic precursors.

91. A method according to claim 67, wherein said method comprises performing assays configured to detect at least four markers selected from the group consisting of adrenomedullin, angiotensinogen, apolipoprotein C1, big endothelin-1, NT-proBNP, proBNP, BNP₇₉₋₁₀₈, BNP, BNP₃₋₁₀₈, complement C3a, calcitonin, caspase-3, CCL19, CCL20, CCL23, CCL26, CCL4, CCL5, CCL8, creatine kinase-BB, C-reactive protein, CXCL13, CXCL16, CXCL6, CXCL5, CXCL9, cystatin C, D-Dimer, sDR6, glutathione-S-transferase A, HMG-1, intestinal fatty acid binding protein, IGFBP-1, IL-10, IL-1β, IL-1RA, IL-22, IL-2sRa, IL-6, IL-8, L-FABP, MCP-1, macrophage migration inhibitory factor, matrix metalloproteinase 9, myeloperoxidase, myoglobin, NGAL, PAI-1, placental growth factor, protein C (activated), protein C (latent), protein C (total), pulmonary surfactant protein A, pulmonary surfactant protein B, pulmonary surfactant protein D, PTEN, RAGE, sICAM1, sphingosine kinase I, tissue factor, TIMP-1, TNF-α, TNF-R1a, TNF-sR14, sTNFRSF3, sTNFRSF7, sTNFRSF11A, sTREM-1, TREM-1sv, uPAR, UCRP, and VCAM-1, or their biosynthetic precursors.

92. A method of diagnosing SIRS in a subject, differentiating causes of SIRS in a subject, or assigning a prognostic risk of one or more future clinical outcomes to a subject suffering from SIRS, the method comprising:
performing one or more assays configured to detect one or more markers selected from the group consisting of angiotensinogen, apolipoprotein C1, CCL20, CXCL5, CXCL9, L-FABP, placental growth factor, sTNFRSF3, sTNFRSF7, and UCRP, or markers related thereto on a blood, serum, or plasma sample obtained from said subject to provide one or more assay results; and
correlating the assay result(s) to the presence or absence of SIRS in the subject, or to the presence or absence of sepsis, severe sepsis, septic shock, or MODS in the subject, or to the prognostic risk of one or more clinical outcomes for the subject.

93. A method according to claim 92, wherein the method further comprises performing one or more assays configured to detect one or more markers selected from the group consisting of adrenomedullin, big endothelin-1, BNP, proBNP, NT-proBNP, CCL5, CCL19, CCL23, CK-MB, complement C3a, creatinine, CXCL13, CXCL16, cystatin C, D-dimer, HSP-60, sICAM-1, IL-1ra, IL-2sRA, IL-6, IL-10, lactate, MCP-1, myoglobin, myeloperoxidase, NGAL, procalcitonin, active protein C, latent protein C, total protein C, serum amyloid A, tissue factor, TNF-R1a, TREM-1, sTNFRSF11A, TIMP-1, and uPAR, or markers related thereto on a blood, serum, or plasma sample obtained from said subject to provide one or more additional assay results;
and said correlating step comprises correlating the assay result(s) and the additional assay result(s) to the presence or absence of SIRS in the subject, or to the presence or absence of sepsis, severe sepsis, septic shock, or MODS in the subject, or to the prognostic risk of one or more clinical outcomes for the subject.

94. A method of diagnosing SIRS in a subject, differentiating causes of SIRS in a subject, or assigning a prognostic risk of one or more future clinical outcomes to a subject suffering from SIRS, the method comprising:
performing one or more assays configured to detect one or markers selected from the group consisting of activated protein C, BNP₇₉₋₁₀₈, CCL4, CXCL6, sDR6, glutathione-S-transferase A, intestinal fatty acid binding protein, placental growth factor, IL2sRA, sphingosine kinase I, sTREM-1, TREM-1sv, and uPAR on one or more samples obtained from said subject to generate one or more assay results; and
correlating the assay results to the presence or absence of SIRS in the subject, or to the presence or absence of sepsis, severe sepsis, septic shock, or MODS in the subject, or to the prognostic risk of one or more clinical outcomes for the subject.

95. A method according to claim 94, wherein the method of differentiating causes of SIRS differentiates between sepsis and severe sepsis or septic shock.

96. A method according to claim 94, wherein the method of differentiating causes of SIRS differentiates between sepsis or severe sepsis and septic shock.

97. A method according to claim 94, wherein the method comprises performing one or more assays configured to detect one or more additional markers not recited in claim 1 to generate one or more additional assay results, and wherein the correlating step comprises correlating the assay results and the additional assay results to the presence or absence of SIRS in the subject, or to the presence or absence of sepsis, severe sepsis, septic shock, or MODS in the subject, or to the prognostic risk of one or more clinical outcomes for the subject.

98. A method according to claim 97, wherein the one or more additional markers are selected from the group consisting of markers related to blood pressure regulation, markers related to inflammation, markers related to apoptosis, and markers related to coagulation and hemostasis.

99. A method according to claim 94, wherein the subject is a human.

100. A method according to claim 94, wherein the one or more sample(s) is(are) selected from the group consisting of blood, serum, and plasma.

101. A method according to claim 94, wherein the assay(s) is(are) immunoassay(s).

102. A method according to claim 94, wherein the method comprises performing one or more assays configured to detect one or more additional markers selected from the group consisting of atrial natriuretic factor, B-type natriuretic peptide, a marker related to B-type natriuretic peptide, C-type natriuretic peptide, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin to generate one or more additional assay results, and wherein the correlating step comprises correlating the assay results and the additional assay results to the presence or absence of SIRS in the subject, or to the presence or absence of sepsis, severe sepsis, septic shock, or MODS in the subject, or to the prognostic risk of one or more clinical outcomes for the subject.

103. A method according to claim 94, wherein the method comprises performing one or more assays configured to detect one or more additional markers selected from the group consisting of acute phase reactants, TNFRSF3, TNFRSF7, TNFRSF11A, LIGHT, CCL16, CXCL5, CXCL9, MMP7, vascular cell adhesion molecule, intercellular adhesion molecule-1, intercellular adhesion molecule-2, intercellular adhesion molecule-3, C-reactive protein, HMG-1, IL-1β, IL-6, IL-8, interleukin-1 receptor agonist, monocyte chemotactic protein-1, caspase-3, lipocalin-type prostaglandin D synthase, mast cell tryptase, eosinophil cationic protein, KL-6, haptoglobin, tumor necrosis factor α, tumor necrosis factor β, fibronectin, macrophage migration inhibitory factor, and vascular endothelial growth factor to generate one or more additional assay results, and wherein the correlating step comprises correlating the assay results and the additional assay results to the presence or absence of SIRS in the subject, or to the presence or absence of sepsis, severe sepsis, septic shock, or MODS in the subject, or to the prognostic risk of one or more clinical outcomes for the subject.

104. A method according to claim 103, wherein the acute phase reactants are selected from the group consisting of hepcidin, HSP-60, HSP-65, HSP-70, S-FAS ligand, asymmetric dimethylarginine, matrix metalloproteins 11, 3, and 9, defensin HBD 1, defensin HBD 2, serum amyloid A, oxidized LDL, insulin like growth factor, transforming growth factor β, an inter-α-inhibitor, e-selectin, hypoxia-inducible factor-1α, inducible nitric oxide synthase, intracellular adhesion molecule, lactate dehydrogenase, monocyte chemoattractant peptide-1, n-acetyl aspartate, prostaglandin E2, receptor activator of nuclear factor ligand, TNF receptor superfamily member 1A, and cystatin C.

105. A method according to claim 94, wherein the method comprises performing one or more assays configured to detect one or more additional markers selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, tissue factor, and thrombus precursor protein to generate one or more additional assay results, and wherein the correlating step comprises correlating the assay results and the additional assay results to the presence or absence of SIRS in the subject, or to the presence or absence of sepsis, severe sepsis, septic shock, or MODS in the subject, or to the prognostic risk of one or more clinical outcomes for the subject.

106. A method according to claim 94, wherein the method comprises performing one or more assays configured to detect one or more additional markers selected from the group consisting of BNP, pro-BNP, and NT-proBNP to generate one or more additional assay results, and wherein the correlating step comprises correlating the assay results and the additional assay results to the presence or absence of SIRS in the subject, or to the presence or absence of sepsis, severe sepsis, septic shock, or MODS in the subject, or to the prognostic risk of one or more clinical outcomes for the subject.

107. A method according to claim 94, wherein the method provides a prognostic risk of mortality.

108. A method according to claim 94, wherein the method comprises performing an immunoassay configured to detect activated protein C.

109. A method according to claim 94, wherein the method comprises performing an immunoassay configured to detect BNP₇₉₋₁₀₈.

110. A method according to claim 94, wherein the method comprises performing an immunoassay configured to detect CCL4.

111. A method according to claim 94, wherein the method comprises performing an immunoassay configured to detect CXCL6.

112. A method according to claim 94, wherein the method comprises performing an immunoassay configured to detect sDR6.

113. A method according to claim 94, wherein the method comprises performing an immunoassay configured to detect glutathione-S-transferase A.

114. A method according to claim 94, wherein the method comprises performing an immunoassay configured to detect intestinal fatty acid binding protein.

115. A method according to claim 94, wherein the method comprises performing an immunoassay configured to detect placental growth factor.

116. A method according to claim 94, wherein the method comprises performing an immunoassay configured to detect IL2sRA.

117. A method according to claim 94, wherein the method comprises performing an immunoassay configured to detect sphingosine kinase I.

118. A method according to claim 94, wherein the method comprises performing an immunoassay configured to detect sTREM-1

119. A method according to claim 94, wherein the method comprises performing an immunoassay configured to detect TREM-1sv.

120. A method according to claim 94, wherein the method comprises performing an immunoassay configured to detect uPAR.
